(19) United States
(12) Patent Application Publication
(10) Pub. No.: US 2005/0090504 A1
(43) Pub. Date: Apr. 28, 2005

(54) Piperidinyl and piperazinyl compounds substituted with bicyclo-heterocyclylalkyl groups useful as CCR3 receptor antagonists

(76) Inventors: Leyi Gong, San Mateo, CA (US); Robert Stephen Wilhelm, Los Altos, CA (US)

Correspondence Address: ROCHE PALO ALTO LLC;PATENT LAW DEPT. M/S A2-250, 3431 HILLVIEW AVENUE, PALO ALTO, CA, 94304, US

(21) Appl. No.: 10/972,564

(22) Filed: Oct. 25, 2004

Related U.S. Application Data
(60) Provisional application No. 60/514,296, filed on Oct. 24, 2003.

(51) Int. Cl. A61K 31/519; A61K 31/496; C07D 43/02

(52) U.S. Cl. 514/252.17; 514/253.06; 514/254.06; 514/254.05; 544/284; 544/363; 544/368; 544/370; 514/254.02

(57) ABSTRACT

Compounds having the Formula (I), 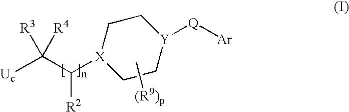
are useful as CCR3 receptor antagonists, wherein Ar is aryl or heteroaryl; Q is —C(═O)— or C₁₋₂alkylene; X is N(⁺)R^{9a}, or N; Y is CR^{9b}, or N; R² is hydrogen or alkyl; R³ and R⁴ are as defined in the specification; U_{c} is a mono- or bicyclic group as defined in the specification; n is 0 or 1; and p is 0, 1, 2, 3 or 4.

### CROSS REFERENCE TO RELATED APPLICATIONS

This application claims the benefit of priority to U.S. Ser. No. 60/514,296 filed Oct. 24, 2003, which is hereby incorporated by reference in its entirety.

### FIELD OF THE INVENTION

The invention relates to certain disubstituted piperidinyl and piperazinyl compounds, in which one of the substituents is a bicyclo-heterocyclylalkyl group, that are useful as CCR-3 receptor antagonists, as well as pharmaceutical compositions containing them and methods for their use.

### BACKGROUND INFORMATION

Tissue eosinophilia is a feature of a number of pathological conditions such as asthma, rhinitis, eczema and parasitic infections (see Bousquet, J. et al., N. Eng. J. Med. 323: 1033-1039 (1990) and Kay, A. B. et al., Br. Med. Bull. 48:51-64 (1992)). In asthma, eosinophil accumulation and activation are associated with damage to bronchial epithelium and hyperresponsiveness to constrictor mediators. Chemokines such as RANTES, eotaxin, and MCP-3 are known to activate eosinophils (see Baggiolini, M. et al., Immunol. Today, 15:127-133 (1994), Rot, A. M. et al., J. Exp. Med. 176, 1489-1495 (1992) and Ponath, P. D. et al., J. Clin. Invest., Vol. 97, No. 3, pp. 604-612 (1996)). However, unlike RANTES and MCP-3 which also induce the migration of other leukocyte cell types, eotaxin is selectively chemotactic for eosinophils (see Griffith-Johnson, D. A. et al., Biochem. Biophys. Res. Commun. Vol. 197, 1167 (1993), and Jose, P. J. et al., Biochem. Biophys. Res. Commun., Vol. 207, 788 (1994)). Specific eosinophil accumulation was observed at the site of administration of eotaxin whether by intradermal or intraperitoneal injection or aerosol inhalation (see Griffith-Johnson, D. A. et al., Biochem. Biophys. Res. Commun., 197:1167 (1993); Jose, P. J. et al., J Exp. Med. 179, 881-887 (1994); Rothenberg, M. E. et al., J. Exp. Med., 181, 1211 (1995), and Ponath, P. D., J. Clin. Invest., Vol. 97, No. 3, 604-612 (1996)).

Glucocorticoids such as dexamethasone, methprednisolone and hydrocortisone have been used for treating many eosinophil-related disorders, including bronchial asthma (R. P. Schleimer et al., Am. Rev. Respir. Dis., 141, 559 (1990)). The glucocorticoids are believed to inhibit IL-5 and IL-3 mediated eosinophil survival in these diseases. However, prolonged use of glucocorticoids can lead to side effects in patients such as glaucoma, osteoporosis, and growth retardation (see Hanania, N. A. et al., J. Allergy and Clin. Immunol., Vol. 96, 571-579 (1995) and Saha, M. T. et al., Acta Paediatrica, Vol. 86, No. 2, 138-142 (1997)). It is desirable to have an alternative means of treating eosinophil-related diseases without incurring these undesirable side effects.

The CCR-3 receptor has been identified as a major chemokine receptor that eosinophils use for their response to eotaxin, RANTES and MCP-3. When transfected into a murine pre-beta lymphoma line, CCR-3 bound eotaxin, RANTES and MCP-3 conferred chemotactic responses on these cells to eotaxin, RANTES and MCP-3 (see Ponath, P. D. et al., J. Exp. Med., 183, 2437-2448 (1996)). The CCR-3 receptor is expressed on the surface of eosinophils, T-cells (subtype Th-2), basophils and mast cells and is highly selective for eotaxin. Studies have shown that pretreatment of eosinophils with an anti-CCR-3 mAb completely inhibits eosinophil chemotaxis to eotaxin, RANTES and MCP-3 (see Heath, H. et al., J. Clin. Invest., Vol. 99, No. 2, 178-184 (1997)). U.S. patent application Ser. No. 10/034,034, filed Dec. 19, 2001, assigned to the present assignee, and U.S. Pat. Nos. 6,140,344, 6,166,015, 6,323,223, 6,339,087, issued to the assignee herein, each describe compounds that are CCR-3 antagonists, and EP application EP903349, published Mar. 24, 1999, discloses CCR-3 antagonists that inhibit eosinophilic recruitment by chemokine such as eotaxin.

Each of the patents and patent applications identified herein is incorporated herein by reference as if set forth at length.

### SUMMARY OF THE INVENTION

The present invention is directed to piperdinyl and piperizinyl compounds useful as CCR3 receptor antagonists which are capable of inhibiting the binding of eotaxin to the CCR-3 receptor and thereby provide a means of combating eosinophil induced diseases, such as asthma.

In a first aspect, this invention provides compounds of Formula (I): 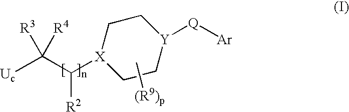
wherein:
Ar is aryl or heteroaryl;
Q is —C(=O)— or C₁₋₂alkylene;
X is N or N⁺R^{9a} Z⁻;
Y is CR^{9a} or N;
Z⁻ is a pharmaceutically acceptable anion;
R² is hydrogen or alkyl;
R³ and R⁴ are, independently of each other, hydrogen, alkyl, substituted alkyl, alkenyl, cycloalkyl, aryl, heteroaryl, heterocyclyl, heteroalkyl, -(alkylene)-C(═O)—Z¹, or -(alkylene)-C(O)₂Z¹, wherein Z¹ is alkyl, haloalkyl, alkoxy, haloalkoxy, hydroxy, amino, alkylamino, aryl, arylalkyl, aryloxy, arylalkyloxy, heteroaryl, or heteroaryloxy;
U_{c} is selected from one of (S), (T), (V), and (W), 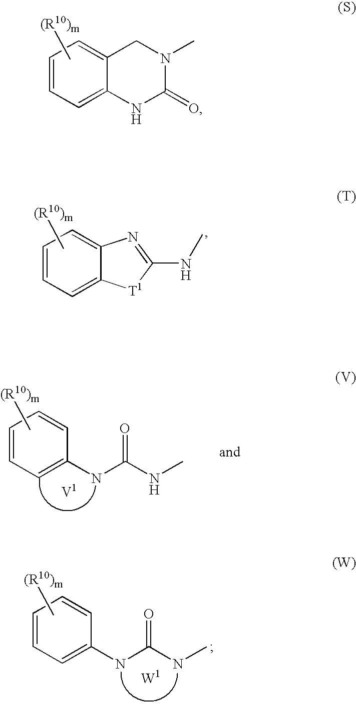
   wherein T¹ is O, S, or NR⁵, wherein R⁵ is selected from hydrogen, alkyl, substituted alkyl, cycloalkyl, and heterocyclyl; and V¹ and W¹ define an optionally substituted five-to-six membered heterocyclic ring; provided, however, that when U^{c} is T and T¹ is S, then R³ and R⁴ are not both hydrogen, and provided that when U^{c} is T and T¹ is S, then at least one of R³ and R⁴ is not hydrogen, and provided that when both X and Y are N, Uc is not T;
R⁹ is attached to any available carbon atom of the piperidinyl or piperazinyl ring and is selected from the group consisting of hydroxy, C₁₋₄ alkoxy, oxo (═O), halogen, cyano, halo C₁₋₄alkyl, halo C₁₋₄alkoxy, and C₁₋₄ alkyl optionally substituted with one to two R¹⁵;
R^{9a} and R^{9b} are selected from the group consisting of hydrogen and C₁₋₄alkyl optionally substituted with one to two R¹⁵;
R¹⁰ is attached to any available carbon atom of the benzo or phenyl ring and at each occurrence is independently selected from the group consisting of C₁₋₄ alkyl, substituted C₁₋₄ alkyl, hydroxy, C₁₋₈ alkoxy, halogen, cyano, C₁₋₈ haloalkoxy, amino, alkylamino, or a heterocyclyl, heteroaryl, C₃₋₇ cycloalkyl, or phenyl in turn optionally substituted with one to three R¹⁶;
R¹⁵ at each occurrence is independently selected from the group consisting of hydroxy, C₁₋₄ alkoxy, halo, cyano, trifluoromethyl, trifluoromethoxy, amino, and alkylamino;
R¹⁶ at each occurrence is independently selected from the group consisting of C₁₋₄ alkyl, hydroxy, C₁₋₄ alkoxy, halo, cyano, trifluoromethyl, trifluoromethoxy, amino, and alkylamino;
m is 0, 1, 2, 3, or 4;
n is 0 or 1;
p is 0, 1, 2, 3 or 4; and,
pharmaceutically-acceptable salts thereof.

The invention also relates to pharmaceutical compositions containing compounds of Formula (I), above, and methods of treating CCR-3 receptor mediated diseases, such as asthma, rhinitis or eczema, by administration of a therapeutically-effective amount of a compound of Formula (I), to a patient in need of treatment thereof.

### DETAILED DESCRIPTION OF THE INVENTION

Unless otherwise stated, the following terms used in the specification and claims have the meanings given below.

“Alkyl” means a linear saturated monovalent hydrocarbon radical of one to eight carbon atoms or a branched saturated monovalent hydrocarbon radical of three to eight carbon atoms, e.g., methyl, ethyl, propyl, 2-propyl, n-butyl, iso-butyl, tert-butyl, pentyl, and the like. A “lower alkyl” is an alkyl group having one to four carbon atoms.

“Alkenyl” means a linear monovalent hydrocarbon radical of two to eight carbon atoms or a branched monovalent hydrocarbon radical of three to eight carbon atoms, containing at least one double bond, e.g., ethenyl, propenyl, and the like.

“Alkynyl” means a linear monovalent hydrocarbon radical of two to eight carbon atoms or a branched monovalent hydrocarbon radical of three to eight carbon atoms, containing at least one triple bond, e.g., ethynyl, propynyl, and the like.

“Alkylene” means a linear saturated bivalent hydrocarbon radical of one to eight carbon atoms or a branched saturated bivalent hydrocarbon radical of three to eight carbon atoms, e.g., methylene, ethylene, 2,2-dimethylethylene, 2-methylpropylene, pentylene, and the like. A “lower alkylene” is said bivalent radical having one to four carbon atoms.

“Alkenylene” means a linear bivalent hydrocarbon radical of two to eight carbon atoms or a branched bivalent hydrocarbon radical of three to eight carbon atoms having at least one double bond, e.g., methenylene, ethenylene, 2,2-dimethylethenylene, 2-methylpropylene, pentylene, and the like. A “lower alkenylene” is said bivalent radical having two to four carbon atoms.

“Substituted alkyl” means an alkyl group having one, two or three substituents selected from the group consisting of acyl, acylamino, hydroxy, C₁₋₈ alkoxy, halo C₁₋₈ alkoxy, cyano, amino, alkylamino, halo C₁₋₈ alkyl, halo, C₁₋₈ alkoxycarbonyl, C₁₋₈ alkylsulfonyl, C₁₋₈ alkylsulfinyl, C₁₋₈ alkylthio, aryl, C₃₋₇cycloalkyl, heteroaryl and/or heterocyclyl, as defined herein. A substituted lower alkyl is an alkyl of one to four carbon atoms having one to three substituents selected from those recited for substituted alkyl, preferably from hydroxy, halo, lower alkoxy, cyano, and haloalkoxy.

When the term “alkyl” is used as a suffix following another term, as in “phenylalkyl,” or “hydroxyalkyl,” this is intended to refer to an alkyl group, as defined above, being substituted with one to two substituents (preferably one substituent) selected from the other, specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl)-3-hydroxypropyl, and so forth. Accordingly, as used herein, the term “hydroxyalkyl” is used to define a subset of heteroalkyl groups defined below.

“Acyl” means a radical —C(═O)R, where R is hydrogen, C₁₋₈ alkyl, C₃₋₇ cycloalkyl, C₃₋₇ cycloalkyl C₁₋₈ alkyl, phenyl, or phenyl C₁₋₈ alkyl, wherein the alkyl, cycloalkyl, cycloalkylalkyl, and phenylalkyl groups are as defined herein. Representative examples include, but are not limited to formyl, acetyl, cyclohexylcarbonyl, cyclohexylmethylcarbonyl, benzoyl, benzylcarbonyl, and the like.

“Acylamino” means a radical —NR'C(═O)R, where R′ is hydrogen or alkyl, and R is hydrogen, alkyl, cycloalkyl, cycloalkylalkyl, phenyl or phenylalkyl, wherein the alkyl, cycloalkyl, cycloalkylalkyl, and phenylalkyl groups are as defined herein. Representative examples include, but are not limited to formylamino, acetylamino, cylcohexylcarbonylamino, cyclohexylmethyl-carbonylamino, benzoylamino, benzylcarbonylamino, and the like.

“Alkoxy ” means a radical —OR, where R is an alkyl as defined herein e.g., methoxy, ethoxy, propoxy, butoxy and the like. A “lower alkoxy” is an alkoxy group wherein the alkyl (R) group has one to four carbon atoms.

When the term “oxy” is used as a suffix following another specifically-named group, as in “aryloxy”, “heteroaryloxy,” or “arylalkyloxy”, this means that an oxygen atom is present as a linker to the other, specifically-named group. Thus, for example, “aryloxy” refers to the group —O—R, wherein R is aryl; “heteroaryloxy” refers to the group —O—R′, wherein R′ is heteroaryl.

“Alkoxycarbonyl” means a radical —C(═O)R, where R is alkoxy is as defined herein.

“Alkylamino” means a radical —NHR or —NRR where R is selected from an C₁₋₈ alkyl, C₃₋₇ cycloalkyl or C₃₋₇ cycloalkyl C₁₋₈ alkyl group as defined herein. Representative examples include, but are not limited to methylamino, ethylamino, isopropylamino, cyclohexylamino, and the like.

“Alkylsulfonyl” means a radical —S(O)₂R, where R is an C₁₋₈ alkyl, C₃₋₇cycloalkyl or C₃₋₇ cycloalkyl C₁₋₈ alkyl group as defined herein, e.g., methylsulfonyl, ethylsulfonyl, propylsulfonyl, butylsulfonyl, cyclohexylsulfonyl and the like.

“Alkylsulfinyl” means a radical —S(O)R, where R is an C₁₋₈ alkyl, C₃₋₇cycloalkyl or C₃₋₇ cycloalkyl C₁₋₈ alkyl group up as defined herein e.g., methylsulfinyl, ethylsulfinyl, propylsulfinyl, butylsulfinyl, cyclohexylsulfinyl and the like.

“Alkylthio” means a radical —SR where R, is an alkyl as defined above e.g., methylthio, ethylthio, propylthio, butylthio, and the like. Mercapto is —SH.

“Aryl” means a monocyclic or bicyclic aromatic hydrocarbon radical which is optionally substituted with one, two or three substituents selected from the group consisting of C₁₋₈ alkyl, heteroalkyl, acyl, acylamino, amino, alkylamino, C₁₋₈ alkylthio, alkylsulfinyl, alkylsulfonyl, —SO₂NR′R″ (where R′ and R″ are independently hydrogen or C₁₋₈ alkyl), C₁₋₈ alkoxy, C₁₋₈ haloalkoxy, C₁₋₈ alkoxycarbonyl, carbamoyl, hydroxy, halo, nitro, cyano, mercapto, methylenedioxy, ethylenedioxy, acyl C₁₋₈ alkyl, acylamino C₁₋₈ alkyl, hydroxy C₁₋₈ alkyl, alkoxy C₁₋₈ alkyl, halo C₁₋₈ alkoxy C₁₋₈ alkyl, C₁₋₈ alkoxycarbonylalkyl, C₁₋₈ alkylsulfonyl C₁₋₈ alkyl, C₁₋₈ alkylsulfinyl C₁₋₈ alkyl, C₁₋₈ alkylthio C₁₋₈ alkyl, or an optionally-substituted phenyl as defined below. More specifically the term aryl includes, but is not limited to, phenyl, chlorophenyl, dichlorophenyl, fluorophenyl, methoxyphenyl, methylphenyl, dimethylphenyl, methylmethoxyphenyl, 1-naphthyl, 2-naphthyl, and so forth.

“Carbamoyl” refers to a group —C(═O)NRR′, wherein R and R″ are independently selected from hydrogen, C₁₋₈ alkyl, substituted C₁₋₈ alkyl, C₃₋₇cycloalkyl, or heterocyclyl.

“Cycloalkyl” refers to a saturated monovalent cyclic hydrocarbon radical of three to seven ring carbons e.g., cyclopropyl, cyclobutyl, cyclohexyl, 4-methylcyclohexyl, and the like, and further includes such rings having a carbon-carbon bridge of one, two, or three bridgehead carbon atoms, and/or having a second ring fused thereto, with the understanding that in such cases the point of attachment will be to the non-aromatic carbocyclic ring moeity. Thus, the term “cycloalkyl” includes such rings as cyclopropyl, cyclopentyl, cyclopentenyl, cyclohexyl, cyclohexenyl, and the like. Additionally, one or two carbon atoms of a cycloalkyl group may optionally contain a carbonyl oxygen group, e.g., one or two atoms in the ring may be a moiety of the formula —C(═O)—.

A “substituted cycloalkyl” is a cycloalkyl group as defined above having one to four (preferably one to two) substituents independently selected from the group of substituents recited above for aryl.

“Halo” means fluoro, chloro, bromo, or iodo, preferably fluoro and chloro.

“Haloalkyl” means alkyl substituted with one or more same or different halo atoms, e.g., —CHF₂, —CF₃, —CH₂CF₃, —CH₂CCl₃, and the like.

“Haloalkoxy” means a group OR, wherein R is haloalkyl as defined above. Thus, it includes such groups as —O—CHF₂, —O—CF₃, and the like.

“Heteroaryl” means a monocyclic or bicyclic radical of 5 to 12 ring atoms having at least one aromatic ring containing one, two, or three ring heteroatoms selected from N, O, or S, the remaining ring atoms being C, with the understanding that when the heteroaryl group is a bicyclic system, the point of attachment to the heteroaryl group will be to an aromatic ring containing at least one heteroatom. The heteroaryl ring is optionally substituted with one, two, three or four substituents, preferably one or two substituents, independently selected from C₁₋₈ alkyl, C₁₋₈ heteroalkyl, acyl, acylamino, amino, C₁₋₈ alkylamino, C₁₋₈ alkylthio, C₁₋₈ alkylsulfinyl, C₁₋₈ alkylsulfonyl, —SO₂NR′R″ (where R′ and R″ are independently hydrogen or C₁₋₈ alkyl), C₁₋₈ alkoxy, C₁₋₈ haloalkoxy, C₁₋₈ alkoxycarbonyl, carbamoyl, hydroxy, halo, nitro, cyano, mercapto, methylenedioxy, ethylenedioxy, acyl C₁₋₈ alkyl, acylamino C₁₋₈ alkyl, C₁₋₈ hydroxyalkyl, C₁₋₈ alkoxy C₁₋₈ alkyl, C₁₋₈ haloalkoxy C₁₋₈ alkyl, cyanoalkyl, aminoalkyl, alkylaminoalkyl, haloalkyl, haloalkyl(alkyl), C₁₋₈ alkoxycarbonyl C₁₋₈ alkyl, C₁₋₈ alkylsulfonyl C₁₋₈ alkyl, C₁₋₈ alkylsulfinyl C₁₋₈ alkyl, and C₁₋₈ alkylthio C₁₋₈ alkyl, or optionally-substituted phenyl as defined below. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyrimidinyl, 5-(3,4-dimethoxyphenyl)-pyrimidin-2-yl, 5-(4-methoxyphenyl)-pyrimidin-2-yl, 5-(3,4-methylenedioxyphenyl)-pyrimidin-2-yl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, benzimidazolyl, benzisoxazolyl, benzothienyl and derivatives thereof.

“Heteroalkyl” means an alkyl radical as defined herein wherein one, two or three hydrogen atoms have been replaced with a substituent independently selected from the group consisting of —OR^{a}, —NR^{b}R^{c}, and —S(O)ₙR^{d} (where n is an integer from 0 to 2), with the understanding that the point of attachment of the heteroalkyl radical is through a carbon atom, wherein R^{a} is hydrogen, acyl, C₁₋₈ alkyl, C₃₋₇cycloalkyl, or C₃₋₇cycloalkyl C₁₋₈ alkyl; R^{b} and R^{c} are independently of each other hydrogen, acyl, C₁₋₈ alkyl, C₃₋₇cycloalkyl, or C₃₋₇ cycloalkyl C₁₋₈ alkyl; and when n is 0, R^{d} is hydrogen, C₁₋₈ alkyl, C₃₋₇ cycloalkyl, or C₃₋₇ cycloalkyl C₁₋₈ alkyl, and when n is 1 or 2, R^{d} is C₁₋₈ alkyl, C₃₋₇ cycloalkyl, C₃₋₇ cycloalkyl C₁₋₈ alkyl, amino, acylamino, or alkylamino. Representative examples include, but are not limited to, 2-hydroxyethyl, 3-hydroxypropyl, 2-hydroxy-1-hydroxymethylethyl, 2,3-dihydroxypropyl, 1-hydroxymethylethyl, 3-hydroxybutyl, 2,3-dihydroxybutyl, 2-hydroxy-1-methylpropyl, 2-aminoethyl, 3-aminopropyl, 2-methylsulfonylethyl, aminosulfonylmethyl, aminosulfonylethyl, aminosulfonylpropyl, methylaminosulfonylmethyl, methylaminosulfonylethyl, methylaminosulfonylpropyl, and the like.

“Heterocyclyl” means a saturated or unsaturated non-aromatic cyclic radical of 3 to 8 ring atoms in which one or two ring atoms are heteroatoms selected from 0, S(O), (where n is an integer from 0 to 2), and NR^{x}, the remaining ring atoms being carbon atoms wherein each R^{x} is independently hydrogen, C₁₋₈ alkyl, acyl, C₁₋₈ alkylsulfonyl, aminosulfonyl, (C₁₋₈ alkylamino)sulfonyl, carbamoyl, (C₁₋₈ alkylamino)carbonyl, (carbamoyl) C₁₋₈ alkyl, or (C₁₋₈ alkylamino)carbonyl C₁₋₈ alkyl. The heterocyclyl ring may be optionally substituted with one, two, or three substituents independently selected as valence permits from C₁₋₈ alkyl, C₁₋₈ haloalkyl, C₁₋₈ heteroalkyl, halo, nitro, cyano, cyano C₁₋₈ alkyl, hydroxy, C₁₋₈ hydroxyalkyl, amino, alkylamino, —(X)ₙ—C(═O)R (where X is O or NR′, n is 0 or 1, R is hydrogen, C₁₋₈ alkyl, C₁₋₈ haloalkyl, hydroxy, C₁₋₈ alkoxy, amino, or alkylamino); C₁₋₈alkylene-C(═O)R (where R is hydrogen, C₁₋₈ alkyl, C₁₋₈ haloalkyl, hydroxy, C₁₋₈ alkoxy, amino, or alkylamino); and/or —S(O)ₙR^{d} (where n is an integer from 0 to 2, and R^{d} is hydrogen, C₁₋₈ alkyl, C₁₋₈ haloalkyl, C₃₋₇ cycloalkyl, C₃₋₇ cycloalkyl C₁₋₈ alkyl, amino, alkylamino, or hydroxy C₁₋₈ alkyl, provided that R^{d} is not hydrogen when n is 1 or 2). More specifically, the term heterocyclyl includes, but is not limited to, tetrahydropyranyl, piperidino, N-methylpiperidin-3-yl, piperazino, N-methylpyrrolidin-3-yl, 3-pyrrolidino, morpholino, thiomorpholino, thiomorpholino-1-oxide, thiomorpholino-1,1-dioxide, tetrahydrothiophenyl-S,S-dioxide, pyrrolinyl, imidazolinyl, and derivatives thereof.

“Leaving group” has the meaning conventionally associated with it in synthetic organic chemistry, i.e., an atom or a group capable of being displaced by a nucleophile and includes halo (such as chloro, bromo, and iodo), alkanesulfonyloxy, arenesulfonyloxy, alkylcarbonyloxy (e.g., acetoxy), arylcarbonyloxy, mesyloxy, tosyloxy, trifluoromethanesulfonyloxy, aryloxy (e.g., 2,4-dinitrophenoxy), methoxy, N,O-dimethylhydroxylamino, and the like.

“Optional” or “optionally” means that the subsequently described event or circumstance may but need not occur, and that the description includes instances where the event or circumstance occurs and instances in which it does not. For example, “aryl optionally substituted with an alkyl” means that the alkyl may but need not be present, and the description includes situations where the aryl group is mono- or disubstituted with an alkyl group and situations where the aryl group is not substituted with the alkyl group.

“Optionally-substituted phenyl” or “optionally substituted pyrimidinyl group” means a phenyl group or a pyrimidinyl group which is optionally substituted with one, two or three substituents (preferably one to two) independently selected from C₁₋₈ alkyl, heteroalkyl, acyl, acylamino, amino, alkylamino, C₁₋₈ alkylthio, C₁₋₈ alkylsulfinyl, C₁₋₈ alkylsulfonyl, —SO₂NR′R″ (where R′ and R″ are independently hydrogen or C₁₋₈ alkyl), C₁₋₈ alkoxy, C₁₋₈ haloalkoxy, C₁₋₈ alkoxycarbonyl, hydroxy, halo, nitro, cyano, mercapto, acyl C₁₋₈ alkyl, acylamino C₁₋₈ alkyl, C₁₋₈ hydroxyalkyl, C₁₋₈ alkoxy C₁₋₈ alkyl, C₁₋₈ haloalkoxy C₁₋₈ alkyl, cyano C₁₋₈ alkyl, amino C₁₋₈ alkyl, C₁₋₈ alkylamino C₁₋₈ alkyl, C₁₋₈ haloalkyl, C₁₋₈ haloalkyl(C₁₋₈ alkyl), C₁₋₈ alkoxycarbonyl C₁₋₈ alkyl, C₁₋₈ alkylsulfonyl C₁₋₈ alkyl, C₁₋₈ alkylsulfinyl C₁₋₈ alkyl, and C₁₋₈ alkylthio C₁₋₈ alkyl. More specifically the term includes, but is not limited to, phenyl, chlorophenyl, fluorophenyl, bromophenyl, methylphenyl, ethylphenyl, methoxyphenyl, cyanophenyl, 4-nitrophenyl, 4-trifluoromethylphenyl, 4-chlorophenyl, 3,4-difluorophenyl, 2,3-dichlorophenyl, 3-methyl4-nitrophenyl, 3-chloro-4-methylphenyl, 3-chloro-4-fluorophenyl or 3,4-dichlorophenyl and the derivatives thereof. An “optionally-substituted pyrimidinyl” means a pyrimidinyl ring optionally having one, two, or three (preferably one or two) substituents selected from those recited for optionally-substituted phenyl.

“Pharmaceutically acceptable excipient” means an excipient that is useful in preparing a pharmaceutical composition that is generally safe, non-toxic and neither biologically nor otherwise undesirable, and includes excipients that are acceptable for veterinary use as well as human pharmaceutical use. A “pharmaceutically acceptable excipient” as used in the specification and claims includes both one and more than one such excipient.

“Pharmaceutically-acceptable salt” of a compound means a salt that is generally safe, non-toxic and neither biologically nor otherwise undesirable, and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, and the like.

The term “pharmaceutically acceptable anion” as used herein means refers to the conjugate base of an inorganic acid or an organic acid used to form a pharmaceutically acceptable salt as defined above. When as acid releases a proton, the remaining species retains an electron pair to which the proton was formerly attached. This species can, in principle, reacquire a proton and is referred to as a conjugate base.

A “prodrug” of a compound of formula (I) herein refers to any compound which releases an active drug according to Formula I in vivo when such prodrug is administered to a mammalian subject. Prodrugs of a compound of Formula I are prepared by modifying one or more functional group(s) present in the compound of Formula I in such a way that the modification(s) may be cleaved in vivo to release the compound of Formula I. Prodrugs include compounds of Formula I wherein a hydroxy, amino, or sulfhydryl group in a compound of Formula I is bonded to any group that may be cleaved in vivo to regenerate the free hydroxyl, amino, or sulfhydryl group, respectively. Examples of prodrugs include, but are not limited to, esters (e.g., acetate, formate, and benzoate derivatives), carbamates (e.g., N,N-dimethylaminocarbonyl) of hydroxy functional groups in compounds of Formula I, and the like.

“Protecting group” refers to a grouping of atoms that when attached to a reactive group in a molecule masks, reduces or prevents that reactivity. Examples of protecting groups can be found in T. W. Greene and P. G. M. Wuts, Protective Groups in Organic Chemistry, (Wiley, 2^{nd} ed. 1991) and Harrison and Harrison et al., Compendium of Synthetic Organic Methods, Vols. 1-8 (John Wiley and Sons, 1971-1996). Representative amino protecting groups include, formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like. Representative hydroxy protecting groups include those where the hydroxy group is either acylated or alkylated such as benzyl, and trityl ethers as well as alkyl ethers, tetrahydropyranyl ethers, trialkylsilyl ethers and allyl ethers.

“Treating” or “treatment” of a disease includes: (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

Compounds that have the same molecular Formula but differ in the nature or sequence of bonding of their atoms or the arrangement of their atoms in space are termed “isomers.” Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, if a carbon atom is bonded to four different groups, a pair of enantiomers is possible. An enantiomer can be characterized by the absolute configuration of its asymmetric center and is described by the R— and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th ed., J. March, John Wiley and Sons, New York, 1992).

### PREFERRED EMBODIMENTS

While the Summary of the Invention sets forth the broadest definition of the invention, certain compounds of Formula (I) are preferred.

For example, preferred compounds are compounds of Formula (Ia), 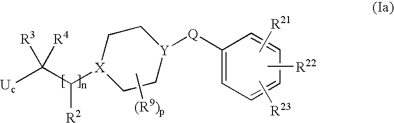
wherein,
X is N or N⁺R^{9a}Z⁻;
Y is N or CR^{9b};
Z is a pharmaceutically acceptable anion;
Q is CH₂;
U_{c} is selected from one of (S), (T), (V), and (W), 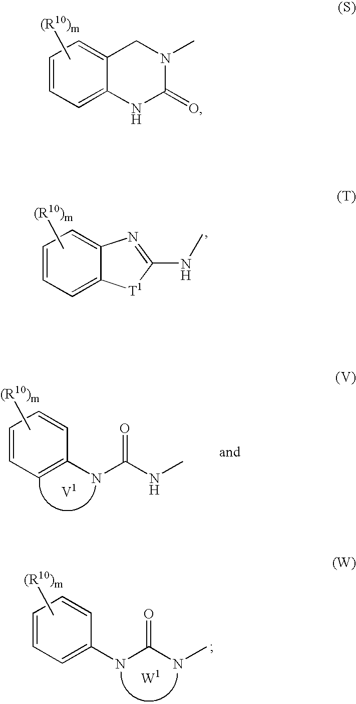
   wherein T¹ is O, S, or NR⁵, wherein R⁵is selected from the group consisting of hydrogen, alkyl, substituted alkyl, cycloalkyl, or heterocyclyl; and V¹ and W¹ define an optionally substituted five-to-six membered heterocyclic ring; provided that when U^{c} is T and T¹ is S, then at least one of R³ and R⁴ is not hydrogen, and provided that when both X and Y are N, Uc is not T;
R² and R³ are hydrogen;
R⁴ is hydrogen, alkyl, hydroxyalkyl, or alkoxyalkyl;
R⁹ is selected from the group consisting of lower alkyl, hydroxy, lower alkoxy and oxo (═O);
R^{9a}is lower alkyl;
R^{9b}is selected from the group consisting of hydrogen, methyl, and ethyl;
R²¹, R²², and R²³ are attached to any available carbon atom of the phenyl ring and are independently selected from the group consisting of hydrogen, lower alkyl, lower alkoxy, halogen, cyano, trifluoromethyl, trifluoromethoxy, C₁₋₄alkylsulfonyl, amino, or alkylamino;
n is 1;
p is 0, 1, or 2; and,
and pharmaceutically acceptable salts thereof.

More preferred are compounds of Formula (la), as defined immediately above, wherein,
R⁴ is methyl, ethyl, 1-methylethyl, isopropyl, 1-hydroxyethyl or 2-hydroxyethyl;
R⁹ is selected from methyl, ethyl, oxo (═O), and hydroxy;
R^{9a}is lower alkyl;
R^{9b}is selected from the group consisting of hydrogen, methyl, and ethyl; and
p is 0 or 1.

In compounds of Formula (Ia), above, preferably R²¹ is hydrogen, and R²² and R²³ are selected from hydrogen, halogen, methyl, and methoxy. More preferred are compounds wherein R²¹, R²², and R²³ and the phenyl ring to which they are attached form 4-chlorophenyl or 3,4-dichlorophenyl.

According to another aspect of the invention, a preferred group of compounds are those compounds of

Formula (I) or (Ia), wherein U^{c} is T, and R⁴ is methyl, ethyl, 1-methylethyl, isopropyl, 1-hydroxyethyl or 2-hydroxyethyl.

According to another aspect of the invention, a preferred group of compounds are those compounds of Formula (I) or (Ia), wherein Q is —CH₂—.

According to another aspect of the invention, a preferred group of compounds are those compounds of Formula (I) or (Ia), wherein R² is hydrogen; and R³ and R⁴ are, independently of each other, hydrogen, alkyl, hydroxyalkyl, or alkoxyalkyl.

According to another aspect of the invention, a preferred group of compounds are those compounds of Formula (I) or (Ia), wherein R⁹ is selected from methyl, ethyl, hydroxy, methoxy, oxo (═O), halo or cyano; and R^{9a} and R^{9b} are selected from hydrogen, methyl and ethyl.

According to another aspect of the invention, a preferred group of compounds are those compounds of Formula (I) or (Ia), wherein n is 1.

According to another aspect of the invention, a preferred group of compounds are those compounds of Formula (I) or (Ia), wherein p is 0.

According to another aspect of the invention, a preferred group of compounds are those compounds of Formula (Ia), above, wherein Y is N.

According to another aspect of the invention, a preferred group of compounds are those compounds of Formula (I) or (Ia), wherein U_{c} is (IIIa).

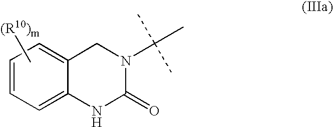

R¹⁰ is selected from lower alkyl, halogen, cyano, and lower alkoxy; and m is 0, 1,or2.

According to another aspect of the invention, a preferred group of compounds are those compounds of Formula (I) or (Ia), wherein U_{c} is (IIIb);

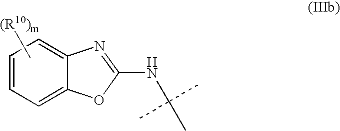

R¹⁰ is selected from the group consisting of lower alkyl, halogen, cyano, and lower alkoxy; and m is 0, 1, or 2.

According to another aspect of the invention, a preferred group of compounds are those compounds of Formula (I) or (Ia), wherein U^{c} is (IIIc);

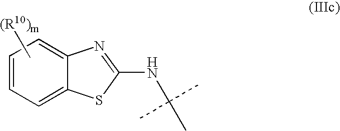

R¹⁰ is selected from the group consisting of lower alkyl, halogen, cyano, and lower alkoxy; and m is 0, 1, or 2.

According to another aspect of the invention, a preferred group of compounds are those compounds of Formula (I) or (Ia), wherein U^{c} is (IIId)

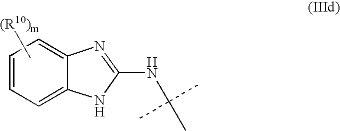

R¹⁰ is selected from the group consisting of lower alkyl, halogen, cyano, and lower alkoxy; and m is 0, 1, or 2.

According to another aspect of the invention, a preferred group of compounds are those compounds of Formula (I) or (Ia), wherein U_{c} is (IIIe); 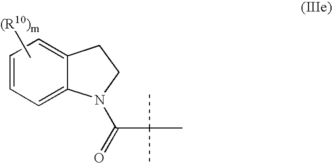
R¹⁰ is selected from the group consisting of lower alkyl, halogen, cyano, and lower alkoxy; and m is 0, 1, or 2.

According to another aspect of the invention, a preferred group of compounds are those compounds of Formula (I) or (Ia), wherein U^{c} is (IIIf);

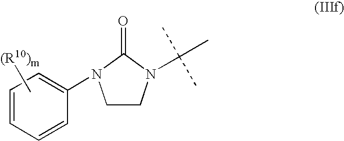

R¹⁰ is selected from the group consisting of lower alkyl, halogen, cyano, and lower alkoxy; and m is 0, 1, or 2.

Other combinations of preferred groups, and/or particularly preferred groups, may form still other groups of preferred compounds. For example, also preferred are compounds having the Formula (Ia): 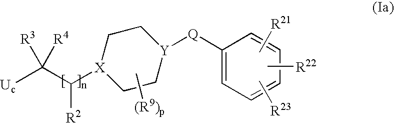
wherein,
X is N or N⁺R^{9a}Z⁻;
Y is N or CR^{9b};
Z is a pharmaceutically acceptable anion;
R² and R³ are hydrogen;
R⁴ is methyl, ethyl, 1-methylethyl, isopropyl, 1-hydroxyethyl or 2-hydroxyethyl;
R⁹ is selected from the group consisting of methyl, ethyl, hydroxy, methoxy, oxo (═O), halo, and cyano;
R^{9a} is lower alkyl;
R^{9b} is hydrogen, methyl or ethyl;
R²¹, R²², and R²³ are attached to any available carbon atom of the phenyl ring and are independently selected from the group consisting of hydrogen, lower alkyl, lower alkoxy, halogen, cyano, trifluoromethyl, trifluoromethoxy, C₁₋₄alkylsulfonyl, amino, and alkylamino.
U^{c} is selected from one of,
   wherein R¹⁰ is selected from lower alkyl, halogen, cyano, and lower alkoxy; and 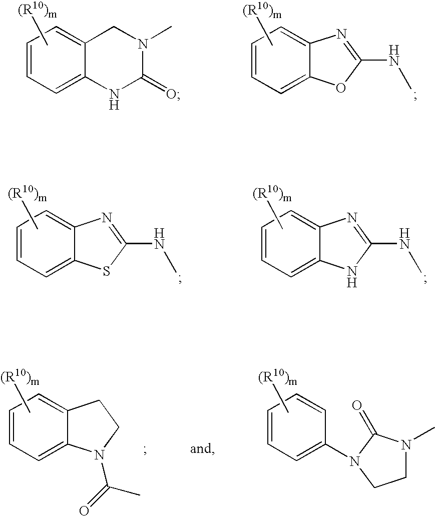
m is 0, 1, or 2;
n is 1; and,
p is 0 or 1.

Other more preferred embodiments are compounds as immediately defined above wherein Q is CH₂.

Even more preferred are compounds as immediately defined above, wherein
R²¹, R²², and R²³, and the phenyl ring to which they are attached, form 4-chlorophenyl or 3,4-dichlorophenyl.

### Utility

The compounds of the invention are CCR-3 receptor antagonists and inhibit eosinophil recruitment by CCR-3 chemokines such as RANTES, eotaxin, MCP-2, MCP-3 and MCP4. Compounds of this invention and compositions containing them are useful in the treatment of eosinophil-induced diseases including inflammatory or allergic diseases, such as inflammatory bowel diseases (e.g., Crohn's disease and ulcerative colitis); psoriasis and inflammatory dermatoses (e.g., dermatitis and eczema), as well as, respiratory allergic diseases such as asthma, allergic rhinitis, hypersensitivity lung diseases, hypersensitivity pneumonitis, and eosinophilic pneumonias (e.g., chronic eosinophilic pneumonia).

### Dosing and Administration

In general, the compounds of this invention can be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors.

Therapeutically effective amounts of compounds of Formula (I) may range from approximately 0.01-20 mg per kilogram body weight of the recipient per day; preferably about 0.1-10 mg/kg/day. Thus, for administration to a 70 kg person, the dosage range would most preferably be about 7 mg to 0.7 g per day.

In general, compounds of this invention will be administered as pharmaceutical compositions by any one of the following routes: oral, transdermal, inhalation (e.g., intranasal or oral inhalation) or parenteral (e.g., intramuscular, intravenous or subcutaneous) administration. A preferred manner of administration is oral using a convenient daily dosage regimen which can be adjusted according to the degree of affliction. Compositions can take the form of tablets, pills, capsules, semisolids, powders, sustained release formulations, solutions, suspensions, liposomes, elixirs, or any other appropriate compositions. Another preferred manner for administering compounds of this invention is inhalation. This is an effective means for delivering a therapeutic agent directly to the respiratory tract for the treatment of diseases such as asthma and other similar or related respiratory tract disorders (see, e.g., U.S. Pat. No. 5,607,915).

The choice of formulation depends on various factors such as the mode of drug administration and the bioavailability of the drug substance. For delivery via inhalation, the compound can be formulated as liquid solutions or suspensions, aerosol propellants or dry powder and loaded into a suitable dispenser for administration. There are three types of pharmaceutical inhalation devices—nebulizer inhalers, metered-dose inhalers (MDI) and dry powder inhalers (DPI). Nebulizer devices produce a stream of high velocity air that causes the therapeutic agents (which has been formulated in a liquid form) to spray as a mist which is carried into the patient's respiratory tract. MDI's typically have the formulation packaged with a compressed gas. Upon actuation, the device discharges a measured amount of therapeutic agent by compressed gas, thus affording a reliable method of administering a set amount of agent. DPI's administer therapeutic agents in the form of a free flowing powder that can be dispersed in the patient's inspiratory air-stream during breathing by the device. In order to achieve a free flowing powder, the therapeutic agent is formulated with an excipient, such as lactose. A measured amount of the therapeutic is stored in a capsule form and is dispensed to the patient with each actuation. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability. The compositions are comprised of a compound of Formula (I) in combination with at least one pharmaceutically-acceptable excipient, as defined above. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

For liposomal formulations of the drug for parenteral or oral delivery the drug and the lipids are dissolved in a suitable organic solvent e.g. tert-butanol, cyclohexane (1% ethanol). The solution is lyophilized and the lipid mixture is suspended in an aqueous buffer and allowed to form a liposome. If necessary, the liposome size can be reduced by sonification. (see Frank Szoka, Jr. and Demetrios Papahadjopoulos, “Comparative Properties and Methods of Preparation of Lipid Vesicles (Liposomes)”, Ann. Rev. Biophys. Bioeng., 9:467-508 (1980), and D. D. Lasic, “Novel Applications of Liposomes”, Trends in Biotech., 16:467-608, (1998)).

Other suitable pharmaceutical excipients and their formulations are described in Remington's Pharmaceutical Sciences, edited by E. W. Martin (Mack Publishing Company, 18th ed., 1990).

The level of the compound in a formulation can vary within the full range employed by those skilled in the art. Typically, the formulation will contain, on a weight percent (wt %) basis, from about 0.01-99.99 wt % of a compound of Formula (I) based on the total formulation, with the balance being one or more suitable pharmaceutical excipients. Preferably, the compound is present at a level of about 1-80 wt %. Representative pharmaceutical formulations containing a compound of Formula (I) are described below.

### Testing

The CCR-3 antagonistic activity of the compounds of this invention can be measured by in vitro assays such as ligand binding and chemotaxis assays as described in more detail below. In vivo activity can be assayed in the Ovalbumin induced Asthma in Balb/c Mice Model as described in more detail below.

### Abbreviations

For ease of reference, the following abbreviations are used in the Schemes and Examples below:
MeOH=methanol
EtOH=ethanol
EtOAc=ethyl acetate
HOAc=acetic acid
DCE=1,2-dichloroethane
DCM=dichloromethane
DMF=dimethylformamide
EDCI=1-(3-dimethylaminopropyl)-3-ethylcarbodiimide hydrochloride
Et=ethyl
Me=methyl
i-Pr=iso-propyl
PCC=pyridinium chlorochromate
PDC=pyridinium dichromate
TEA or Et₃N=triethylamine
THF=tetrahydrofuran
TFA=trifluoroacetic acid
rt. or RT=room temperature

### General Synthetic Schemes

The compounds of the present invention can be prepared in a number of ways known to one skilled in the art. Preferred methods include, but are not limited to, the general synthetic procedures described below.

The starting materials and reagents used are either available from commercial suppliers such as Aldrich Chemical Co. (Milwaukee, Wis., USA), Bachem (Torrance, Calif., USA), Enika Chemie or Sigma (St. Louis, Mo., USA), Maybridge (Dist: Ryan Scientific, P.O. Box 6496, Columbia, S.C. 92960), etc.; or are prepared by methods known to those skilled in the art following procedures set forth in the literature such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 1992); and Larock's Comprehensive Organic Transformations (VCH Publishers Inc., 1989). These schemes are merely illustrative and various modifications to these schemes can be made and will be suggested to one skilled in the art.

The starting materials and the intermediates of the reaction may be isolated and purified if desired using conventional techniques, including but not limited to filtration, distillation, crystallization, chromatography, and the like. Such materials may be characterized using conventional means, including physical constants and spectral data. In the Schemes, the variables X, Y, Q, Ar, R⁴, R²¹, R²², R²³, p, q, etc., are defined as set forth in the claims.

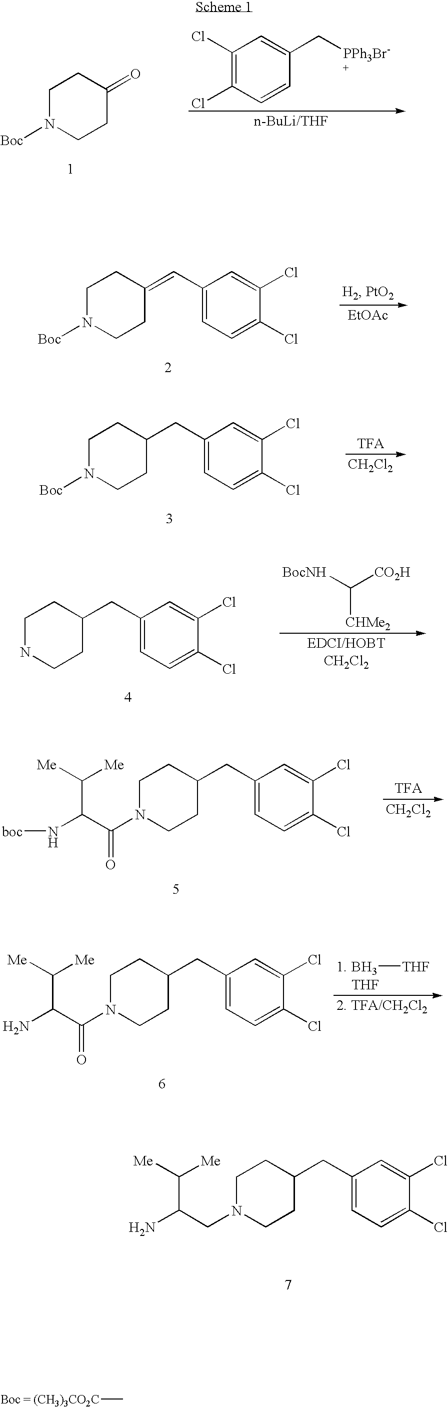

Scheme 1 illustrates a general procedure for preparing piperidinyl intermediates (7), which can then be converted to compounds of Formula (I).. 4-Oxo-piperidine-1-carboxylic acid tert-butyl ester (1) is a suitable starting material to introduce the C-4 substituent. A Wittig condensation with a triphenyl(optionally substituted)benzylphosphonium halide converts the C-4 ketone into a (optionally substituted)phenylalkylidene 2 substituent. Several variants of the Wittig reaction are well known within the art and each can be adapted to the preparation of compounds of the present invention (see, e.g., J. March Advanced Organic Chemistry 4^{th} ed., John Wiley & Sons, New York, 1992, pp. 956-963; A. Maercker, Organic Reactions, John Wiley, New York 1965 v. 14 p 270-490; phosphoryl-stabilized carbanions, W. S. Wadsworth Jr. Organic Reactions John Wiley & Sons, New York, v. 25, 1977, pp. 74-257; Peterson olefination, D. Ager, Organic Reactions John Wiley & Sons, New York, v.38, 1990, pp. 1-224). The Wittig Reaction is generally run by treating a phosphonium salt dissolved or suspended in an inert solvent with a strong base, e.g., n-butyl lithium or lithium diusopropylamide at from −78 to 0° C. The ylide thus formed is added to 1 and stirred at a temperature ranging from −78 to 0° C. until the reaction is completed and the product is purified by standard techniques. The requisite phosphonium salts are prepared by contacting a (optionally substituted) benzyl halide with triphenylphosphine. Benzyl halides are readily available by free radical-induced benzylic halogenation. In the exemplified process 3,4-dichlorotoluene is commercially available from the Sigma-Aldrich (catalog # 16,136-5).

Reduction of the olefin can be readily achieve by a variety of methods including catalytic hydrogenation and removal of the boc protecting group from the nitrogen atom is accomplished by standard protocols (T. W. Greene and P. G. M. Wuts, supra). The boc protecting group is acid sensitive and protocols for cleavage of the boc group typically contact the carbamate with trifluoroacetic acid and methylene chloride at temperatures ranging from 0° C. to room temperature. Alternatively other acids such as hydrochloric acid also will readily cleave the boc group.

Substitution of the piperidinyl nitrogen is readily accomplished by a two-step sequence comprising acylation and reduction of the resulting amide (see also Scheme 2). Acylation of the nitrogen is readily accomplished utilizing the amine acylation protocols developed for peptide synthesis which produce high chemical yields of an amide without racemization of the adjacent chiral center to yield 6.

Prior to carrying out the acylation with an amino acid, the amino group of the amino acid must be protected to prevent undesirable amide formation. Numerous N-protecting groups have been developed which can be selectively cleaved under a variety of conditions. Protection strategies for coupling amino acids have been extensively reviewed (see e.g., M. Bodanszky, Principles of peptide Synthesis, Springer Verlag, New York 1993; P. Lloyd-Williams and F. Albericio Chemical Methods for the Synthesis of Peptides and Proteins CRC Press, Boca Raton, Fla. 1997). These references are incorporated herein in their entirety. The various amino-protecting groups useful in this invention include N-benzyloxy-carbonyl-(cbz), tert-butoxy-carbonyl (Boc), N-formyl- and N-urethane-N-carboxy anhydrides which are all commercially available (SNPE Inc., Princeton, N.J., Aldrich Chemical Co., Milwaukee, Wis., and Sigma Chemical Co., St. Louis, Mo.) N-urethane amino-protected cyclic amino acid anhydrides are also described in the literature (William D. Fuller et al., J. Am. Chem. Soc. 1990 112:7414-7416) which is incorporated herein by reference. While many of these could be effectively employed in the present process, preferred urethane protecting groups include the tert-butoxycarbonyl or the benzyloxycarbonyl.

Protocols for efficient coupling of N-protected amino acids have extensively optimized (M. Bodanszky supra; P. Lloyd-Williams and F. Albericio supra). At least 1 equivalent of the protected amino acid and 1 equivalent of a suitable coupling agent or dehydrating agent, e.g., 1,3-dicyclohexylcarbodiimide or salts of such duimides with basic groups, N-ethyl-N′-(3-(dimethylamino) propyl)carbodiimide hydrochloride, should be employed from the start. Other dehydrating agents such as N,N′-carbonyldiimidazole, trifluoroacetic anhydride, mixed anhydrides, acid chlorides may be used. Numerous additives have been identified which improve the coupling efficiency and limit racemization of the alpha-amino acid including, 1-hydroxybenzotriazole and 3-hydroxy-3,4-dihydro-4-oxo-1,2,3-benzotriazine (W. König and R. Geiger Chem. Ber.1970 788:2024 and 2034), N-hydroxysuccinimide (E. Wunsch and F. Drees, Chem. Ber. 1966 99:110), 1-hydroxy-7-azabenzotriazole (L. A. Carpino J. Am. Chem. Soc. 1993 115:4397-4398). Aminium/uronium- and phosphonium HOBt/HOAt-based coupling reagents have been developed, e.g. based peptide coupling reagents, e.g., 1-benzotriazol-1-yloxy-bis(pyrrolidino)uronium hexafluorophosphate (J. Xu and S. Chen Tetrahedron Lett. 1992 33:647), 1-benzotriazol-1-yloxy-N,N-dimethylmethananiminium hexachloroantimonate (P. Li and J. Xu, Tetrahedron Lett. 1999 40:3606), O-(7-azabenzotriazol-1-yl)-1,1,3,3-tetramethylammoniumuronium hexafluorophosphate (L. A. Carpino, J. Am. Chem. Soc. 1993 115:4397), O-(7-azabenzotriazol-1-yl)-1,1,3,3-bis-(tetramethylene)uronium hexafluorophosphate (A. Erlich et al. Tetrahedron Lett. 1993 34:4781), 2-(3,4-dihydro-4-oxo-1,2,3-benzotriazin-3-yl)-1,1,3,3-tetramethyluronium tetrafluoroborate (R. Knorr et al. Tetrahedron Lett. 1989 30:1927), 7-azobenzotriazolyoxy-tris-(pyrrolidino) hexafluorophosphate (F. Albericio et al., Tetrahedron Lett. 1997 38:4853), 1-benzotriazolyloxy-tris-(dimethylamino)phosphonium hexafluorophosphate (B. Castro et al. Tetrahedron Lett. 1976 14:1219) and, 1-benzotriazoloxy-tris-pyrrolidinophosphonium hexafluorophosphate (J. Coste et al. Tetrahedron Lett. 1990 31:205).

Removal of the boc protecting group in an analogous manner to that described above affords 7 which can be can be converted to the compounds of the present invention. Reduction of 6 is typically carried out with a solution of diborane in THF in a manner well known to those of skill in the art (e.g. the reaction is run under inert conditions with an inert solvent, typically cyclic or acyclic ethers at about −20° C. to 70° C.). Alternate reducing agents are well known in the art (J. March, supra p. 1212-1213; A. G. M. Barrett Reduction of Carboxylic Acid Derivatives to Alcohols, Ethers and Amines in Comprehensive Organic Synthesis vol. 8, I. Fleming (Ed) 1991 248-251). An alternative procedure to the two-step acylation and reduction sequence is direct alkylation of the piperidinyl nitrogen which may be advantageous depending on the nature of the amine and the alkylating agent. (Gibson in The Chemistry of the Amino Group S. Patai (ed), John Wiley, New York, 1968 p. 45-55).

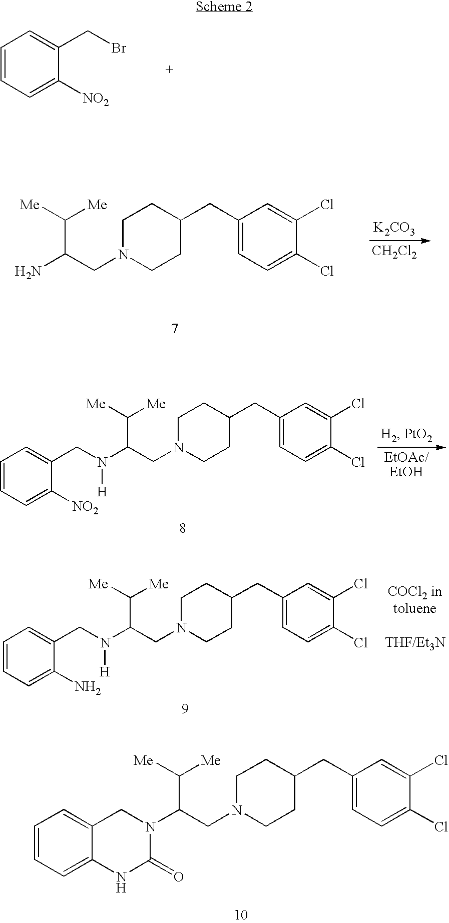

The preparation of 3-{1-[4-(3,4-dichloro-benzyl)-piperidin-1-ylmethyl]-2-methyl-propyl{-3,4-dihydro-1H-quinazolin-2-one (10) shown in Scheme 2 illustrates the primary amine into a cyclic urea, and specifically a 4-dihydro-1H-quinazolin-2-one. Alkylation of amine 7 with 1-bromomethyl-2-nitrobenzene affords 8. Reduction of the nitro group to a primary amine was accomplished by catalytic hydrogenation to yield 9. Alternative procedures for reduction of a nitro group are well know and can also be adapted to the preparation of the compounds of the present invention (J. March, supra, p. 1216-1217). Intramolecular cyclization of the primary and secondary amines with phosgene or a phosgene equivalent such as diumidazole carbonyl afforded the urea 10 (A. F. Katritzky and A. F. Pozharskii Handbook of Heterocyclic Chemistry, 2^{nd} Ed. Pergamon Press, Oxford 2000, p.573; A. F. Hegarty and L. J. Diennen, Functions Containing Carbonyl Groups and Two Heteroatoms other then a Halogen of a Chalcone in Comprehensive Organic Functional Group Transformations, T. L. Gilchrist (ed.) v. 6 chapter 6.16, Pergamon Press, Oxford 1995 pp. 506-507; see pp. 500-501 for corresponding intermolecular process).

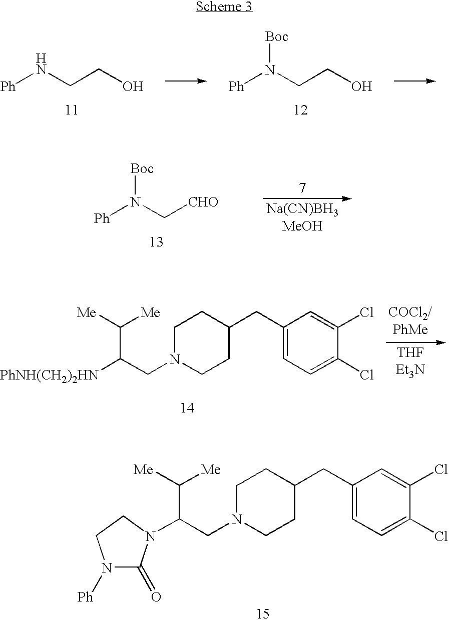

An alternative to the amine acylation/reduction or alkylation sequences to substitute the piperdinyl nitrogen of 4 is reductive amination. Scheme 3 is an adaptation of the process to the synthesis of a 3-phenyl-imidazolidin-2-one. 2-Phenylaminoethanol (11) is treated with di-tert-butyl-dicarbonate to introduce the Boc protecting group and subsequently converted to 13 by oxidation with pyridinium dichromate to afford 13. Reductive amination (R. M. Hutchings and M. K. Hutchings Reduction of C═N to CHNH by Metal Hydrides in Comprehensive Organic Synthesis, vol. 8, I. Fleming (Ed) Pergamon, Oxford 1991 pp. 47-54) of 13 with piperidine 7 affords the triamine 14 which is subjected to intramolecular cyclization with phosgene to yield 1-{1-[4-(3,4-dichloro-benzyl)-piperidin-1-ylmethyl]-2-methyl-propyl}-3-phenyl-imidazolidin-2-one (15). Scheme 4 depicts the phosgene-mediated intermolecular coupling of two amines, 7 and 2,3-dihydroindole to afford urea 16.

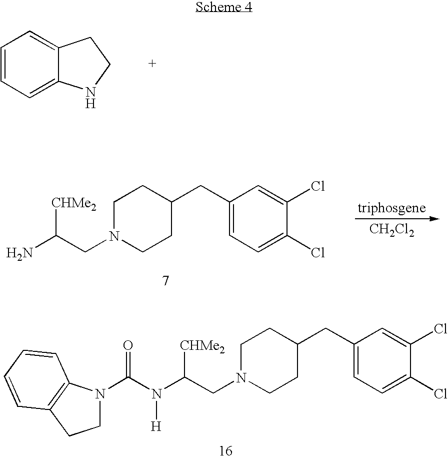

Piperazine derivatives of the present invention can be prepared from the commercially available 1-boc-piperzine (Fluka; catalog number 15502). The unprotected amine can be substituted by direct alkylation of the amine or by an acylation/reduction sequence as described above. (Scheme 5). In the exemplified synthesis the amine is alkylated by 3,4-dichloro-bromomethyl-benzene. Removal of the boc protecting group with acid affords 18b. The N-(2-amino-3-methylbutyl) substituent is incorporated by acylation/reduction analogously to the sequence described in Scheme 1. Coupling of 18a with Boc-NH-Val-OH affords amide 19 which is deprotected by TFA treatment and subsequently reduced with diborane-THF to afford 1-[4-(3,4-dichloro-benzyl)-piperazin-1-ylmethyl]-2-methyl-propylamine (21). Intra-molecular cyclization of the primary amine with phosgene or an equivalent afforded N-carbamoyl, 3,4-dihydro-1H-quinazolin-2-one and imidazolidin-2-one derivatives as previously exemplified in Schemes 2 and 3.

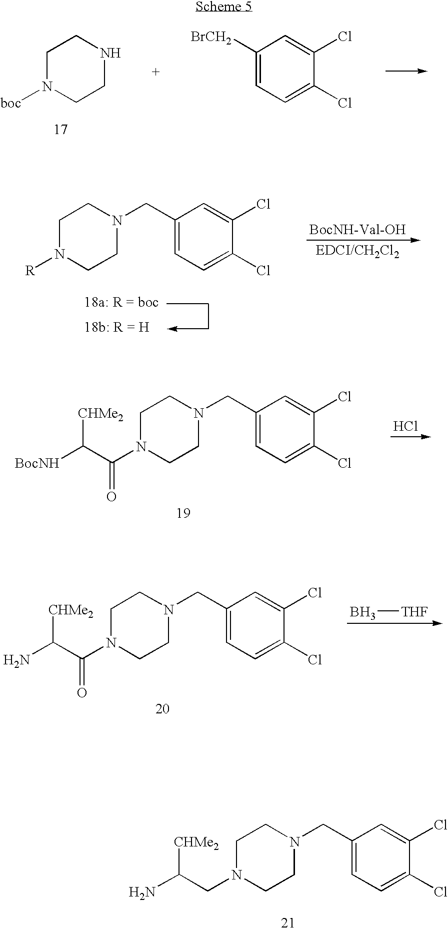

Heterocycle-substituted amines were prepared by contacting 21 with an optionally substituted heterocyclic ring susceptible to attack by nucleophiles. 2-Chlorobenzoxazole derivatives 23 are susceptible to attack by nucleophilic amines with subsequent expulsion of chloride ion to afford 2-aminobenzoxazoles compounds. Reacting 21 with 23 affords benzoxazol-2-yl-{1-[4-(3,4-dichloro-benzyl)-piperazin-1-ylmethyl]-2-methyl-propyl}-amine (24). 2-chloro-benzoxazoles (Scheme 6) are prepared by sequential treatment with potassium ethoxydithiocarbonate and thionyl chloride to afford 23. The preparation of benzoxazoles has been reviewed (G. V. Boyd Comprehensive Heterocyclic Chemistry, K. T. Potts (ed.) v. 6, part 4B pp. 216-227)

Benzothiazoles and benzimidazoles of the present invention can be prepared analogously from benzothiazoles and benzimidazoles from suitable precursors. The synthesis of benzothiazoles and benzimidazoles is well known in the art (Benzothiazoles; J. Metzger, Thiazoles and their Benzo Derivatives in Comprehensive Heterocyclic Chemistry K. T. Potts (ed) v. 6, part 4B, Pergamon Press, Oxford pp. 321-326; A. Dondonni and P. Merino, Comprehensive Heterocyclic Chemistry II v. 3, I. Shinkai (ed) Pergamon Press Oxford, 1996, pp. 431-452; Benzimidazoles, M. R. Grimmett Imidazole and their Benzo Derivatives (iii) Synthesis and Applications in Comprehensive Heterocyclic Chemistry K. T. Potts (ed.) Pergamon Press, Oxford v. 5, pp. 457-496; M. R. Grimmett Imidazole and their Benzo Derivatives (iii) Synthesis and Applications in Comprehensive Heterocyclic Chemistry II, I. Shinkai (ed.) v. 3, Pergamon Press, Oxford, 1996, pp. 185-213).

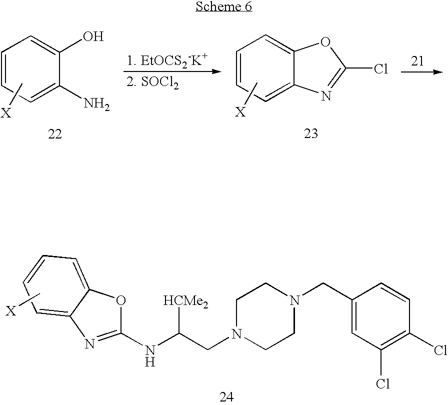

### EXAMPLES

The following preparations and examples are provided to enable those skilled in the art to more clearly understand and to practice the present invention. However, these Examples should not be considered as limiting the scope of the invention, but merely as being illustrative and representative thereof.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. For convenience and consistency, acid addition salts are depicted with the piperidinyl nitrogen protonated. This is not intended to be a limitation and in individual cases protonation or alkylation of other nitrogen atoms can occur and protonation or quaternization of any nitrogen atom could occur and all species are within the scope of the invention.

### Example 1

### 3-{1-[4-(3,4-Dichloro-benzyl)-piperidin-1-ylmethyl]-2-methyl-propyl}-3,4-dihydro-1H-quinazolin-2-one

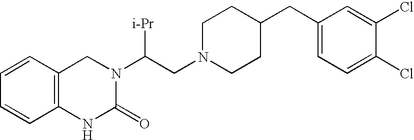 Step 1

n-Butyl lithium (43.2 mL, 2M in pentane, 108 mmol) was slowly added to an ice-cooled suspension of 3,4-dichlorobenzyl triphenylphosphonium bromide (54 g, 108 mmol) (prepared by stirring equimolar amounts of 3,4-dichlorobenzyl bromide and triphenylphosphine in THF at 65° C. overnight) in dry THF (500 mL) under an argon atmosphere. After 15 min., the reaction mixture was allowed to warm to room temperature and then was stirred for an additional 2 h. 1-tert-butoxycarbonyl-4-piperidone (21.4 g, 108 mmol) was added, and the stirring was continued overnight. Hexane (2 l) was added and the reaction was stirred and then filtered. The filtrate was concentrated in vacuo to give 41.8 g of an orange gum. Column purification with silica gel and 70% DCM in hexane, followed by 100% DCM and a gradient of 1% MeOH/DCM through 5% MeOH/DCM gave 1-tert-butoxycarbonyl)-4-(3,4-dichlorobenzylidene)piperidine (29 g) as a light tan oil.

Step 2

Platinum oxide (0.3 g) was added to a solution of 1-(tert-butoxycarbonyl)-3,4-dichlorobenzylidene)piperidine (29 g, 85 mmol) in EtOAc (500 mL), and the mixture was stirred under a hydrogen atmosphere overnight. The reaction mixture was filtered through a CELITE® bed and the filtrate was concentrated to give 1-(tert-butoxycarbonyl)-3,4-dichlorobenzyl)piperidine (30 g) as an oil.

Step 3

TFA (50 mL) was added to a solution of 1-(tert-butoxycarbonyl)-3,4-dichlorobenzyl)piperidine (24 g, 70 mmol) in DCM (150 mL), and the reaction mixture was stirred for 1 h. The solvent was removed in vacuo, followed by addition of EtOAc (200 mL), and the resulting mixture was made basic with 1N aqueous sodium hydroxide. The organic layer was separated, dried over magnesium sulfate, and the solvent was removed in vacuo to give 4-(3,4-dichlorobenzyl)piperidine (17 g) as light brown solid.

Step 4

To a solution of 4-(3,4-dichlorobenzyl)piperidine (23 g, 1.3 eq.) were added D-BOC-Valine (20 g, 82 mmol), EDCI (1-(3-dimethylaminopropyl)-3-ethylcarbodiimide) (20.3 g, 1.3 eq.) and HOBT (benzotriazol-1-ol, 2.2 g, 0.2 eq.). The resulting mixture was stirred at rt. overnight. Volatile was removed and the residue was partitioned between EtOAc and aqueous NaHCO₃. The organic layer was washed with saturated brine and dried over Na₂SO₄. The crude product was purified on a silica gel column with 20% EtOAc in hexane to afford 36 g of {1-[4-(3,4-dichloro-benzyl)-piperidine-1-carbonyl]-2-methyl-propyl}-carbamic acid tert-butyl ester as a white foam.

Step 5

To a solution of {1-[4-(3,4-dichloro-benzyl)-piperidine-1-carbonyl]-2-methyl-propyl}-carbamic acid tert-butyl ester (36 g, 0.08 mol) in 100 mL of CH₂C₁₋₂ was added TFA (35 mL, 0.45 mol). After the mixture was stirred at room temperature for 16 h, the volatile was removed and the residue was partitioned between EtOAc and KOH (20 g) in 100 nL of water. The organic layer was separated and washed with water, brine, and dried over Na₂SO₄. Concentration gave 28 g of 2-amino-1-[4-(3,4-dichloro-benzyl)piperidin-1-yl]-3-methyl-butan-1-one.

Step 6

2-Amino-1-[4-(3,4-dichloro-benzyl)piperidin-1-yl]-3-methyl-butan-1-one (28 g, 0.08 mol) was dissolved in 250 mL of THF and mixed with 500 mL of BH₃-THF (1.0 M). The reaction mixture was heated to reflux for 3h, then allowed to cool to RT, then cooled to an ice bath temperature. The solution was acidified with the dropwise addition of 3N HCl until pH<3. Volatile was removed and the residue was reconstituted in 100 mL of EtOH and 300 mL of 3N HCl. After the resulting mixture was heated to 82° C. for 1.5 h, it was cooled to RT and then basified with NaOH (aq.). The product was extracted with EtOAc and the organic layer was washed with NaCl (sat.) and dried over Na₂SO₄. Column purification on silica gel with 2.5% to 5% of (10% NH₄OH in MeOH) in CH₂Cl₂ gave 24 g of 1-[4-(3,4-dichloro-benzyl)-piperidin-1-ylmethyl]-2-methylpropylamine.

Step 7

2-Nitrobenzyl bromide (69 mg, 1.05 eq.) was mixed up with 1-[4-(3,4-dichloro-benzyl)-piperidin-1-ylmethyl]-2-methylpropylamine (100 mg, 0.3 mmol) in 5 mL of CH₂Cl₂ in the presence of K₂CO₃ (84 mg, 2 eq.). After the mixture was stirred at room temperature overnight, it was quenched with water and extracted with EtOAc. The organic layer was separated, washed with brine, and dried over Na₂SO₄. Column purification on silica gel with 25% acetone, 25% CH₂C₁₂ in hexane gave 100 mg of {1-[4-(3,4-dichloro-benzyl)-piperidin-1-ylmethyl]-2-methyl-propyl}-(2-nitro-benzyl)-amine as an oil.

Step 8

{1-[4-(3,4-Dichloro-benzyl)-piperidin-1-ylmethyl]-2-methyl-propyl}-(2-nitro-benzyl)-amine (90 mg, 0.19 mmol) was reduced under 1 atm of H₂ in EtOH/EtOAc (5 mL/5 mL) in the presence of PtO₂. After stirring for 2h, it was filtered through a CELITE® bed and concentrated to give 89 mg of 2-({1-[4-(3,4-dichloro-benzyl)-piperidin-1-ylmethyl]-2-methyl-propylamino}-methyl)-phenylamine.

Step 9

To a solution of 2-({1-[4-(3,4-dichloro-benzyl)-piperidin-1-ylmethyl]-2-methyl-propylamino{-methyl)-phenylamine (80 mg, 0.18 mmol) in 10 mL of dry THF was added Et₃N (0.094 mL, 3.7 eq.), followed by the addition of 20% phosgene in toluene (0.087 mL, 0.18 mmol). After the mixture was stirred at RT for 2h, the volatile was removed. The residue was partitioned between water and CH₂Cl₂. The organic layer was washed with water, NaCl (sat.) and dried over Na₂SO₄. Column purification with 5% MeOH in CH₂Cl₂ gave 70 mg of the desired product, 3-{1-[4-(3,4-dichloro-benzyl)-piperidin-1-ylmethyl]-2-methyl-propyl}-3,4-dihydro-1H-quinazolin-2-one.

### Example 2

### 1-{1-[4-(3,4-Dichloro-benzyl)-piperidin-1-ylmethyl]-2-methyl-propyl}-3-phenyl-imidazolidin-2-one

Step 1

2-Phenylamino-ethanol (5.0 g, 36 mmol) and di-t-butyl-dicarbonate (1.9 g, 1.5 eq.) in 50 mL of THF 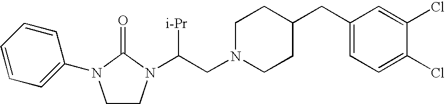
was heated to 55° C. for 7 h. Volatile was then removed in vacuo. The crude product was recrystallized from CH₂Cl₂ and hexane to give 8.1 g of white crystalline material ((2-hydroxy-ethyl)-phenyl-carbamic acid tert-butyl ester).
Step 2

(2-Hydroxyethyl)-phenyl-carbamic acid tert-butyl ester (3.0 g, 13 mmol) was mixed with PDC (5.3 g, 1.1 eq.) in 50 mL of CH₂Cl₂ and stirred at RT for 16 h. The reaction mixture was then diluted with Et₂O, filtered through florisil, and the colorless filtrate was concentrated. The residue was purified on a silica gel column with 15% EtOAc in hexane to give 1.6 g of (2-oxo-ethyl)-phenyl-carbamic acid tert-butyl ester as a colorless oil.

Step 3

A mixture of (2-oxo-ethyl)-phenyl-carbamic acid tert-butyl ester (0.5 g, 2.13 mmol) and 1-[4-(3,4-dichloro-benzyl)-piperidin-1-ylmethyl]-2-methylpropylamine (0.7 g, 1 eq.) in 30 mL of MeOH was stirred with 3 Å molecular sieves (10 g) for 0.5 h. NaCNBH₃ (0.081 g, 0.6 eq.) was then added and the mixture was stirred for another 3 h. The reaction was quenched with a few drops of 3N HCl and filtered through a CELITE® bed. The crude product was purified on a silica gel column with 3% (10% NH₄OH in MeOH) in CH₂Cl₂ to give 0.35 g of N-{1-[4-(3,4-dichloro-benzyl)-piperidin-1-ylmethyl]-2-methyl-propyl}-N′-phenyl-ethane-1,2-diamine.

Step 4

To a solution of N-{1-[4-(3,4-dichloro-benzyl)-piperidin-1-ylmethyl]-2-methyl-propyl}-N′-phenyl-ethane-1,2-diamine (0.2 g, 0.45 mmol) and Et₃N (0.22 mL, 3.5 eq.) in 25 mL of THF was added 20% phosgene in toluene (0.42 mL, 0.85 mmol) dropwise. The solution was stirred for 1 h at RT, and the volatile was removed. The residue was partitioned between EtOAc and NaHCO₃ (aq.), and the organic layer was separated, washed with brine, and dried over Na₂SO₄. Preparative TLC with 5% MeOH, 2.5% hexane in CH₂Cl₂ gave 0.12 g of Example 2, i.e., 1-{1-[4-(3,4-dichloro-benzyl)-piperidin-1-ylmethyl]-2-methyl-propyl}-3-phenyl-imidazolidin-2-one, which was converted to HCl salt.

### Example 3

### Benzothiazol-2-yl-{1-[4-(3,4-dichloro-benzyl)-piperazin-1-ylmethyl]-2-methyl-propyl}-amine

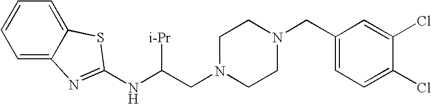 Step 1

3,4-Dichlorobenzyl bromide (35.2 g, 150 mmol) was added to a solution of N-(tert-butoxycarbonyl)piperazine (24.8 g, 130 mmol) and TEA (21 mL, 150 mmol) in DCM (100 mL) over 30 min. After 1h, the reaction mixture was diluted with EtOAc, and the product precipitated out as the hydrochloride salt with addition of 1N aqueous hydrogen chloride solution. The solid product was filtered, washed with water, and then resuspended in EtOAc. Two equivalents of 1N aqueous sodium hydroxide solution was added and the free amine was extracted into EtOAc. The organic layer was separated, dried over magnesium sulfate, filtered, and concentrated to provide 1-(tert-butoxycarbonyl)-4-(3,4-dichlorobenzyl)piperazine (45 g).

Step 2

TFA (75 mL, 0.97 mol) was added to a solution of 1-(tert-butoxycarbonyl)-4-(3,4-dichlorobenzyl)piperazine (45 g, 0.13 mol) in DCM (75 mL). The mixture was stirred for 1 h at room temperature and then made basic with a sodium hydroxide solution. The product was extracted into EtOAc and the organic layer was washed with sodium bicarbonate solution, dried over magnesium sulfate, and concentrated in vacuo to give 1-(3,4-dichlorobenyl)piperazine (35.8 g) as a solid.

Step 3

1-(3-Dimethylaminopropyl)-3-ethylcarbodiimide hydrochloride (5.08 g, 26.5 mmol) was added to a solution of 1-(3,4-dichlorobenzyl)piperazine (5 g, 20.4 mmol) and (D,L)-Boc-valine (5.76 g, 26.5 mmol) in DCM. After 2 h, the product was extracted into EtOAc. The organic layer was washed with sodium bicarbonate solution, dried over magnesium sulfate, filtered and concentrated in vacuo. Column chromatography with hexane/EtOAc (1:1) gave 1-[4-(3,4-dichlorobenzyl)piperazin-1-ylcarbonyl]-N-(tert-butoxycarbonyl)-2-methylpropylamine (5.46 g) as a foam.

Step 4

Ethereal hydrogen chloride solution (80 mL, 80 mmol) was added to a solution of 1-[4-(3,4-dichlorobenzyl)piperazin-1-ylcarbonyl]-N-(tert-butoxycarbonyl)-2-methylpropylamine (4.28 g, 9.64 mmol) in MeOH (50 mL) and the mixture was heated at 70° C. After 2.5 h, the reaction mixture was concentrated and the solid was suspended in ether and filtered to give 1-[4-(3,4-dichlorobenzyl)piperazin-1-ylcarbonyl]-2-methylpropylamine as the hydrochloride salt. The product was dissolved in water, treated with TEA (4 mL) and the free amine was extracted into EtOAc. The EtOAc layer was dried over magnesium sulfate, filtered, and concentrated to give 1-[4-(3,4-dichlorobenzyl)piperazin-1-ylcarbonyl]-2-methylpropylamine (3.2 g) as the free amine.

Step 5

A 1.0 M diborane solution in THF (65.2 mL, 65.2 mmol) was added to a solution of 1-[4-(3,4-dichlorobenzyl)piperazin-1-ylcarbonyl]-2-methylpropylamine (3.2 g, 9.3 mmol) in THF (15 mL). The mixture was heated at reflux under nitrogen for 2 h and then concentrated in vacuo. The residue was dissolved in MeOH, acidified with 6 N hydrogen chloride solution (50 mL), and then reheated to 70° C. After 1 h, the reaction mixture was cooled and basified with a sodium hydroxide solution and the product was extracted into EtOAc. The EtOAc layer was washed with sodium bicarbonate solution, dried over magnesium sulfate, filtered and concentrated to provide 1-[4-(3,4-dichlorobenzyl)piperazin-1-ylmethyl]-2-methylpropylamine (3.53 g) as an oil.

Step 6

To a solution of 2-methylsulfanyl-benzothiazole (1.22, 6.7 mmol) dissolved in 15 mL of acetic acid was added potassium permanganate (1.81 g, 1.7 eq.) in 17 mL of H₂O. The resulting mixture was heated for 30 min and stirred at RT for over 48 h. The reaction was quenched with NaHSO₃, and the pH of the solution was adjusted to 8 with NH₄OH. The reaction was extracted with EtOAc, the EtOAc layer was washed with H₂O, dried over Na₂SO₄ and concentrated to give the desired product, 2-methanesulfonyl-benzothiazole: (M)⁺: 213.

Step 7

2-Methanesulfonylbenzothiazole (0.055 g, 0.25 mmol) and 1-[4-(3,4-dichlorobenzyl)piperazin-1-ylmethyl]-2-methylpropylamine (84 mg, 0.25 mmol) were heated to 130° C. under argon. After 90 min, the mixture was cooled. It was then purified on a silica gel column with 40% EtOAc in hexane first, followed by 1% iso-PrNH₂, 10% MeOH in EtOAc to give benzothiazol-2-yl-{1-[4-(3,4-dichloro-benzyl)-piperazin-1-ylmethyl]-2-methyl-propyl}-amine (39%): (M)⁺=462.

### Example 4

### Benzooxazol-2-yl-{1-[4-(3,4-dichloro-benzyl)-piperazin-1-ylmethyl]-2-methylpropyl}amine

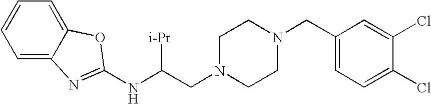

To a solution of 1-[4-(3,4-dichlorobenzyl)piperazin-1-ylmethyl]-2-methylpropylamine (0.108 g, 0.33 mmol) and diisopropylethylamine (0.17 mL, 3eq.) in 1.5 mL of THF was added dropwise 2-chloro-benzooxazole (0.04 mL, 0.36 mmol) in 0.36 mL of THF at 0° C. The resulting mixture was stirred at 0° C. for 2 h and then allowed to warm to RT, where it was stirred for an additional 2 h. Volatile was removed in vacuo and the residue was partitioned between EtOAc and water. The organic layer was washed with brine and dried over sodium sulfate. The crude product was purified on a silica gel column with 40% EtOAc in hexane first, followed by 1% i-PrNH₂, 10% MeOH in EtOAc to give benzooxazol-2-yl-{1-[4-(3,4-dichloro-benzyl)-piperazin-1-ylmethyl]-2-methyl-propyl}amine (85%): (M)⁺=446.

### Examples 5-7

The compounds described in Table 1 were prepared following the procedure described in Example 3, Steps 1-5 and Example 4 above, but substituting BOC-valine with the desired amino acid, i.e., L-BOC-valine (Ex. 5), D-BOC-valine (Ex. 6), and BOC-glycine (Ex. 7).

TABLE 1

| Ex. | | | (MW) | CCR3 |
|---|---|---|---|---|
| No. | Structure | Compound Name | MS m.p. | IC₅₀ |
| 5 | 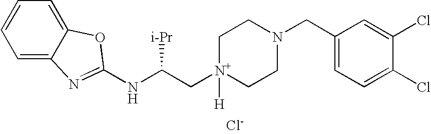 | 1-[(R)-2-(Benzooxazol-2- ylamino)-3-methyl-butyl]-4- (3,4-dichloro-benzyl)- piperazin-1-ium; chlorideRO1164829-001 | 447.41 | |
| 6 | 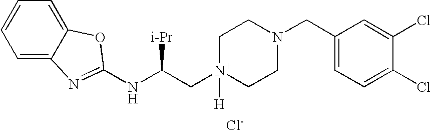 | 1-[(S)-2-(Benzooxazol-2- (ylamino)-3-methyl-butyl]-4- (3,4-dichloro-benzyl)- piperazin-1-ium; chloride RO4002895-001 | 447.41 M⁺=446 | 4.35 |
| 7 | 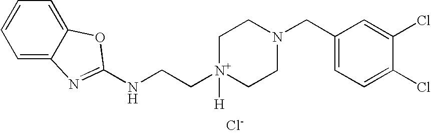 | 1-[2-(Benzooxazol-2- ylamino)-ethyl]-4-(3,4- dichloro-benzyl)-piperazin- 1-ium; chloride RO1164827-001 | 405.33 M⁺=404 | |

### Example 8

### {1-[4-(3,4-Dichloro-benzyl)-piperazin-1-ylmethyl]-2-methyl-propyl}-(6-methoxy-benzooxazol-2-yl)-amine

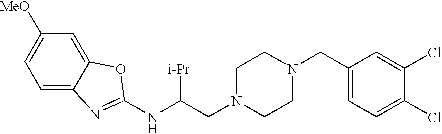 Step 1

2-Amino-5-methoxy-phenol hydrochloride salt (0.203 g, 1.2 mmol) and potassium salt of dithiocarbonic acid O-ethyl ester were dissolved in 4 mL of pyridine and heated to reflux for 2 h. The reaction mixture was cooled to RT and quenched by pouring into 5 mL of ice-cold water. To the mixture, 0.22 mL of conc. HCl was added and stirred for 30 min. The solid was filtered, washed with water, and dried in vacuo overnight. To the above product was added SOCl₂ (0.55 mL, 7.6 mmol) and 2 drops of DMF. After the reaction was heated to 70° C. for 30 min, it was cooled RT. Excess SOCl₂ was removed in vacuo and the residue was purified on a silica gel column with 5% MeOH in CH₂Cl₂ to give {1-[4-(3,4-dichloro-benzyl)-piperazin-1-ylmethyl]-2-methyl-propyl}-(6-methoxy-benzooxazol-2-yl)-amine.

### Example 9

### {1-[4-(3,4-Dichloro-benzyl)-piperazin-1-ylmethyl]-2-methyl-propyl}-(5-methyl-benzooxazol-2-yl)-amine

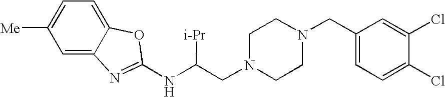 Step 1

To a solution of 2-amino-p-cresol (1.81 g, 0.015 mol) and KOH (1.2 eq. 0.99 g) in 30 mL of EtOH was added methanedithione (18 mL). The resulting mixture was heated to reflux for 18 h. Upon cooling, the volatiles were removed in vacuo and the residue was partitioned between EtOAc and 18 mL of 1N HCl. The organic layer was separated, washed with water, dried over sodium sulfate, and concentrated to give 1.2 g of 5-methyl-3H-benzooxazole-2-thione: (M+H)⁺=165.

Step 2

5-Methyl-3H-benzooxazole-2-thione (0.539 g, 1.64 mmol) and 1-[4-(3,4-dichlorobenzyl)piperazin-1-ylmethyl]-2-methylpropylamine (0.225 g, 1.64 mmol) were dissolved in 1.5 mL of toluene and heated to reflux for 2 h. The reaction mixture was cooled to RT and the volatiles were removed in vacuo. The crude product was purified on a silica gel column with 40% EtOAc in hexane, followed by 1% i-PrOH, 9% MeOH in EtOAc to give 0.25 g of {1-[4-(3,4-dichloro-benzyl)-piperazin-1-ylmethyl]-2-methyl-propyl}-(5-methyl-benzooxazol-2-yl)-amine: m.p. 155.3-156.9° C.; MS: (M+H)⁺=461.

### Examples 10-11

The compounds described in Table 2 were prepared following the procedure described in Example 1, Steps 1-6 and example 3, but substituting BOC-valine with the desired amino acid BOC-glycine (Ex. 10).

TABLE 1

| Ex. | | | (MW)MS |
|---|---|---|---|
| No. | Structure | Compound Name | mp(° C.) |
| 10 | 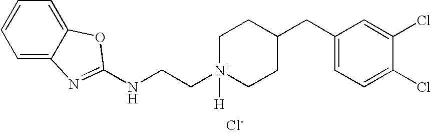 | 1-[2-(Benzooxazol-2- ylamino)-ethyl]-4-(3,4- dichloro-benzyl)- piperidinium; chloride | 404 M⁺=404 217-233 |

### Examples 14-35

Examples 14-35 as described in Table 3 were prepared following the same or similar methods described above for Examples 1 through 11 and illustrated in Schemes 1 through 6.

TABLE 3

| Ex. | | | (MW) |
|---|---|---|---|
| No. | Structure | Compound Name | M+H mp |
| 14 | 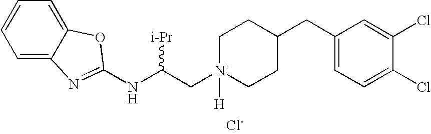 | 1-[2-(Benzooxazol-2- ylamino)-3-methyl-butyl]-4- (3,4-dichloro-benzyl)- piperidinium; chloride | 446.42 446 151-156 |
| 15 | 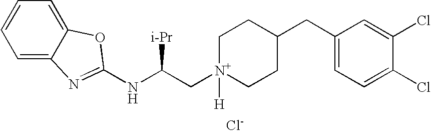 | 1-[(S)-2-(Benzooxazol-2- ylamino)-3-methyl-butyl]-4- (3,4-dichloro-benzyl)- piperidinium; chloride | |
| 17 | 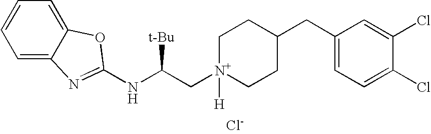 | 1-[(S)-2-(benzooxazol-2- ylamino)-3,3-dimethyl- butyl]-4-(3,4-dichloro- benzyl)-piperidinium; chloride | 460.45 460 253-258 |
| 18 | 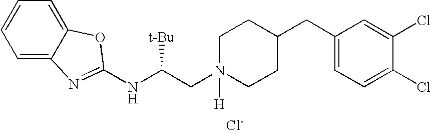 | 1-[(R)-2-(benzooxazol-2- ylamino)-3,3-dimethyl- butyl]-4-(3,4-dichloro- benzyl)-piperidinium; chloride | 460.45 460 |
| 19 | 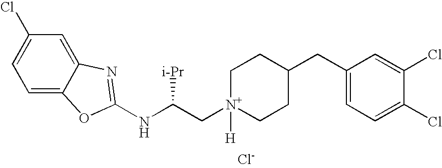 | 1-[(R)-2-(6-chloro- benzooxazol-2-ylamino)-3- methyl-butyl]-4-(3,4- dichloro-benzyl)- piperidinium; chloride | 480.86 480 215-224 |
| 20 | 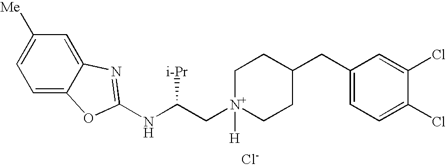 | 4-(3,4-Dichloro-benzyl)-1- [(R)-3-methyl-2-(5-methyl- benzooxazol-2-ylamino)- butyl]-piperidinium; chloride | 460.45 460 141.0-144.5 |
| 21 | 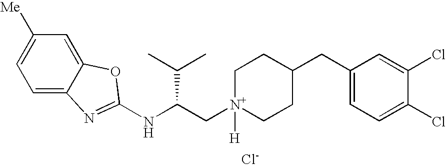 | 4-(3,4-Dichloro-benzyl)-1- [(R)-3-methyl-2-(6-methyl- benzooxazol-2-ylamino)- butyl]-piperidinium; chloride | 460.45 M⁺=459 |
| 22 | 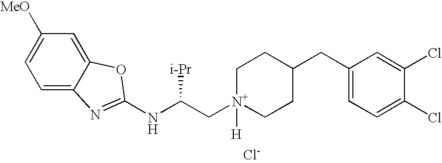 | 4-(3,4-Dichloro-benzyl)-1- [(R)-2-(6-methoxy- benzooxazol-2-ylamino)-3- methyl-butyl]-piperidinium; chloride | 476.45 476 |
| 23 | 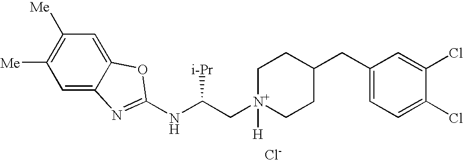 | 4-(3,4-Dichloro-benzyl)-1- [(R)-2-(5,6-dimethyl- benzooxazol-2-ylamino)-3- methyl-butyl]-piperidinium; chloride | 474.47 M⁺=473 145-1533 |
| 24 | 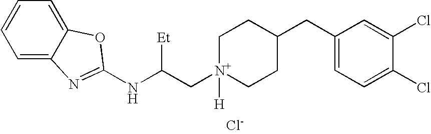 | 1-[2-(Benzooxazol-2- ylamino)-butyl]-4-(3,4- dichloro-benzyl)- pipendinium; chloride | 432.39 432 123-130 |
| 25 | 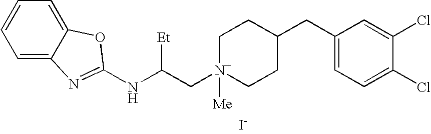 | 1-[2-(Benzooxazol-2- ylamino)-butyl]-4-(3,4- dichloro-benzyl)-1-methyl- piperidinium; iodide | 447.43 M⁺=446 123.0-128.5 |
| 26 | 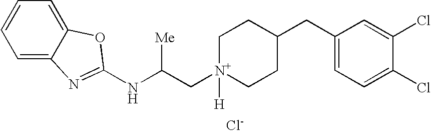 | 1-[2-(Benzooxazol-2- ylamino)-propyl]-4-(3,4- dichloro-benzyl)- piperidinium; chloride | 418.37 418 |
| 27 | 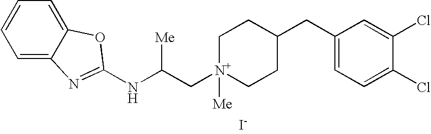 | 1-[2-(Benzooxazol-2- ylamino)-propyl]-4-(3,4- dichloro-benzyl)-1-methyl- piperidinium iodide | 433.40 432 129.0-137.5 |
| 28 | 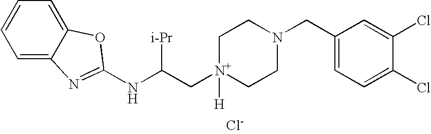 | 1-[2-(Benzooxazol-2- ylamino)-3-methyl-butyl]-4- (3,4-dichloro-benzyl)- piperazin-1-ium; chloride | 447.41 446 |
| 29 | 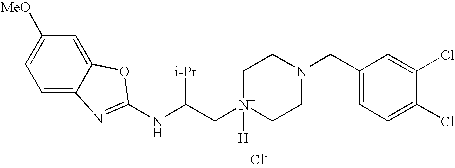 | 4-(3,4-Dichloro-benzyl)-1- [2-(6-methoxy-benzooxazol- 2-ylamino)-3-methyl-butyl]- piperazin-1-ium; chloride | 477.43 476 |
| 30 | 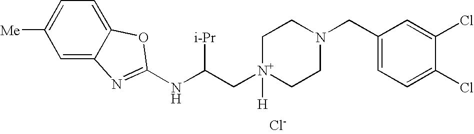 | 4-(3,4-Dichloro-benzyl)-1- [3-methyl-2-(5-methyl- benzooxazol-2-ylamino)- butyl]-piperazin-1-ium; chloride | 461.43 461 |
| 31 | 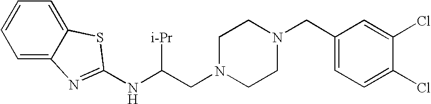 | Benzothiazol-2-yl-{1-[4- (3,4-dichloro-benzyl)- piperazin-1-ylmethyl]-2- methyl-propyl}-amine | 463.47 M⁺=462 |
| 32 | 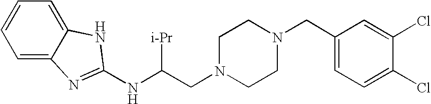 | (1H-Benzoimidazol-2-yl)- {1-[4-(3,4-dichloro-benzyl)- piperazin-1-ylmethyl]-2- methyl-propyl}-amine | 446.42 446 |
| 34 | 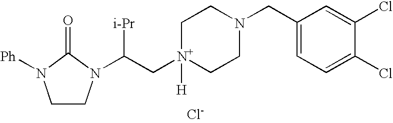 | 4-(3,4-Dichloro-benzyl)-1- [3-methyl-2-(2-oxo-3- phenyl-imidazolidin-1-yl)- butyl]-piperazin-1-ium; chloride | 474.47 474 |
| 35 | 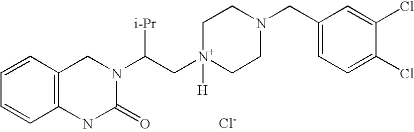 | 4-(3,4-Dichloro-benzyl)-1- [3-methyl-2-(2-oxo-1,4- dihydro-2H-quinazolin-3- yl)-butyl]-piperazin-1-ium; chloride | 460.45 460 |

### Example 36

### Formulation Examples

The following are representative pharmaceutical formulations containing a compound of Formula (I).

Tablet Formulation
The following ingredients are mixed
intimately and pressed into single scored tablets.

| | Quantity per |
|---|---|
| Ingredient | tablet, mg |
| compound of this invention | 400 |
| cornstarch | 50 |
| croscarmellose sodium | 25 |
| lactose | 120 |
| magnesium stearate | 5 |

Capsule Formulation
The following ingredients are mixed
intimately and loaded into a hard-shell gelatin capsule.

| | Quantity per |
|---|---|
| Ingredient | capsule, mg |
| compound of this invention | 200 |
| lactose, spray-dried | 148 |
| magnesium stearate | 2 |

Suspension Formulation
The following ingredients are
mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.5 | g |
| sorbit (70% solution) | 12.85 | g |
| Veegum K (Vanderbilt Co.) | 1.0 | g |
| flavoring | 0.035 | mL |
| colorings | 0.5 | mg |
| distilled water | q.s. to 100 | mL |

Injectable Formulation
The following ingredients
are mixed to form an injectable formulation.

| Ingredient | Amount |
|---|---|
| compound of this invention | 0.2 g |
| sodium acetate buffer solution | 0.4M 2.0 mL |
| HCl (1N) or NaOH (1N) | q.s. to suitable pH |
| water (distilled, sterile) | q.s. to 20 mL |

Liposomal Formulation
The following ingredients are mixed to form a liposomal formulation.

| Ingredient | Amount | |
|---|---|---|
| compound of this invention | 10 | mg |
| L-.alpha.-phosphatidylcholine | 150 | mg |
| tert-BuOH | 4 | mL |

Freeze dry the sample and lyophilize overnight. Reconstitute the sample with 1 mL 0.9% saline solution. Liposome size can be reduced by sonication.

### Example 37

### CCR-3 Receptor Binding Assay—In Vitro

The CCR-3 antagonistic activity of the compounds of the invention was determined by their ability to inhibit the binding of ¹²⁵ I eotaxin to CCR-3 L1.2 transfectant cells (see Ponath, P. D. et al., J. Exp. Med., Vol. 183, 2437-2448, (1996)).

The assay was performed in Costar 96-well polypropylene round bottom plates. Test compounds were dissolved in DMSO and then diluted with binding buffer (50 mM HEPES, 1 mM CaCl.sub.2, 5 mM MgCl₂, 0.5% bovine serum albumin (BSA), 0.02% sodium azide, pH 7.24) such that the final DMSO concentration was 2%. 25 μl of the test solution or only buffer with DMSO (control samples) was added to each well, followed by the addition of 25 μl of ¹²⁵I-eotaxin (100 pmol) (NEX314, New England Nuclear, Boston, Mass.) and 1.5×10⁵ of the CCR-3 L1.2 transfected cells in 25 μl binding buffer. The final reaction volume was 75 μl.

After incubating the reaction mixture for 1 hour at rt., the reaction was terminated by filtering the reaction mixture through polyethylenimine treated Packard Unifilter GF/C filter plate (Packard, Chicago, Ill.). The filters were washed four times with ice cold wash buffer containing 10 mm HEPES and 0.5M sodium chloride (pH 7.2) and dried at 65° C. for approximately 10 minutes. 25 μl/well of Microscint-20® scintillation fluid (Packard) was added and the radioactivity retained on the filters was determined by using the Packard TopCount®. Compounds of this invention were tested and found to have a measurable level of activity in this assay.

| | CCR3 Binding |
|---|---|
| Example | IC₅₀ (μM) |
| 18 | 0.15 |
| 28 | 0.97 |

### Example 38

### Inhibition of Eotaxin Mediated Chemotaxis of CCR-3 L1.2 Transfectanted Cells—In Vitro Assay

The CCR-3 antagonistic activity of the compounds of this invention can be determined by measuring the inhibition of eotaxin mediated chemotaxis of the CCR-3 L1.2 transfectant cells, using a slight modification of the method described in Ponath, P. D. et al., J. Clin. Invest. 97: 604-612 (1996). The assay is performed in a 24-well chemotaxis plate (Costar Corp., Cambridge, Mass.). CCR-3 L1.2 transfectant cells are grown in culture medium containing RPMI 1640, 10% Hyclone® fetal calf serum, 55 mM 2-mercaptoethanol and Geneticin 418 (0.8 mg/mL). 18-24 hours before the assay, the transfected cells are treated with n-butyric acid at a final concentration of 5 mM/1×10⁶ cells/mL, isolated and resuspended at 1×10⁷ cells/mL in assay medium containing equal parts of RPMI 1640 and Medium 199 (M 199) with 0.5% bovine serum albumin.

Human eotaxin suspended in phosphate buffered saline at 1 mg/mL is added to bottom chamber in a final concentration of 100 nm. Transwell culture inserts (Costar Corp., Cambridge, Mass.) having 3 micron pore size are inserted into each well and L1.2 cells (1×10⁶) are added to the top chamber in a final volume of 100 μl. Test compounds in DMSO are added both to the top and bottom chambers such that the final DMSO volume is 0.5%. The assay is performed against two sets of controls. The positive control contained cells with no test compound in the top chamber and only eotaxin in the lower chamber. The negative control contains cells with no test compound in the top chamber and neither eotaxin nor test compound in lower chamber. The plate is incubated at 37° C. After 4 hours, the inserts are removed from the chambers and the cells that have migrated to the bottom chamber are counted by pipetting out 500 μl of the cell suspension from the lower chamber to 1.2 mL Cluster tubes (Costar) and counting them on a FACS for 30 seconds.

### Example 39

### Inhibition of Eotaxin Mediated Chemotaxis of Human Eosinophils—In Vitro Assay

The ability of compounds of the invention to inhibit eotaxin mediated chemotaxis of human eosinophils can be assessed using a slight modification of procedure described in Carr, M. W. et al., Proc. Natl. Acad. Sci. USA, 91: 3652-3656 (1994). Experiments are performed using 24 well chemotaxis plates (Costar Corp., Cambridge, Mass.). Eosinophils are isolated from blood using the procedure described in PCT Application, Publication No. WO 96/22371. The endothelial cells used are the endothelial cell line ECV 304 obtained from European Collection of Animal Cell Cultures (Porton Down, Salisbury, U.K.). Endothelial cells are cultured on 6.5 mm diameter Biocoat.RTM. Transwell tissue culture inserts (Costar Corp., Cambridge, Mass.) with a 3.0 μM pore size. Culture media for ECV 304 cells consists of M199, 10% Fetal Calf Serum, L-glutamine and antibiotics. Assay media consists of equal parts RPMI 1640 and M199, with 0.5% BSA. 24 hours before the assay 2×10⁵ ECV 304 cells are plated on each insert of the 24-well chemotaxis plate and incubated at 37° C. 20 nM of eotaxin diluted in assay medium is added to the bottom chamber. The final volume in bottom chamber is 600 μl. The endothelial coated tissue culture inserts are inserted into each well. 10⁶ eosinophil cells suspended in 100 μl assay buffer are added to the top chamber. Test compounds dissolved in DMSO are added to both top and bottom chambers such that the final DMSO volume in each well was 0.5%. The assay is performed against two sets of controls. The positive control contains cells in the top chamber and eotaxin in the lower chamber. The negative control contains cells in the top chamber and only assay buffer in the lower chamber. The plates are incubated at 37° C. in 5% CO₂/95% air for 1-1.5 hours.

The cells that migrate to the bottom chamber are counted using flow cytometry. 500 μl of the cell suspension from the lower chamber are placed in a tube, and relative cell counts are obtained by acquiring events for a set time period of 30 seconds.

### Example 40

### Inhibition of Eosinophil Influx Into the Lungs of Ovalbumin Sensitized Balb/c Mice by CCR-3 Antagonist—In Vivo Assay

The ability of the compounds of the invention to inhibit leukocyte infiltration into the lungs can be determined by measuring the inhibition of eosinophil accumulation into the bronchioalveolar lavage (BAL) fluid of Ovalbumin (OA)-sensitized balb/c mice after antigen challenge by aerosol. Briefly, male balb/c mice weighing 20-25 g are sensitized with OA (10 μg in 0.2 mL aluminum hydroxide solution) intraperitoneally on days 1 and 14. After a week, the mice are divided into ten groups. Test compound or only vehicle (control group) or anti-eotaxin antibody (positive control group) is administered either intraperitoneally, subcutaneously or orally. After 1 hour, the mice are placed in a Plexiglass box and exposed to OA aerosol generated by a PARISTAR™ nebulizer (PARI, Richmond, Va.) for 20 minutes. Mice which have not been sensitized or challenged are included as a negative control. After 24 or 72 hours, the mice are anesthetized (urethane, approx. 1 g/kg, i.p.), a tracheal cannula (PE 60 tubing) is inserted and the lungs are lavaged four times with 0.3 mL PBS. The BAL fluid is transferred into plastic tubes and kept on ice. Total leukocytes in a 20 μl aliquot of the BAL fluid is determined by Coulter Counter.™. (Coulter, Miami, Fla.). Differential leukocyte counts are made on Cytospin.™. preparations which have been stained with a modified Wright's stain (DiffQuick.™.) by light microscopy using standard morphological criteria.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

1. A compound having the Formula (I): 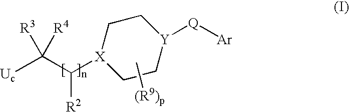
wherein:
Ar is aryl or heteroaryl;
Q is —C(═O)— or C₁₋₂alkylene;
X is N, or N⁺R^{9a} Z⁻; Y is CR^{9a} or N;
Z is a pharmaceutically acceptable anion;
R² is hydrogen or alkyl;
R³ and R⁴ are, independently of each other, hydrogen, C₁₋₈ alkyl, substituted alkyl, C₂₋₈ alkenyl, C₃₋₇ cycloalkyl, aryl, heteroaryl, heterocyclyl, heteroalkyl, —(C₁₋₈ alkylene)-C(═O)-Z¹, or —(C₁₋₈alkylene)-C(O)₂Z¹, wherein Z¹ is C₁₋₈ alkyl, C₁₋₈ haloalkyl, C₁₋₈ alkoxy, C₁₋₈ haloalkoxy, hydroxy, amino, alkylamino, aryl, aryl C₁₋₈ alkyl, aryloxy, aryl C₁₋₈ alkyloxy, heteroaryl, or heteroaryloxy;
U_{c} is selected from one of (S), (T), (V), and (W), 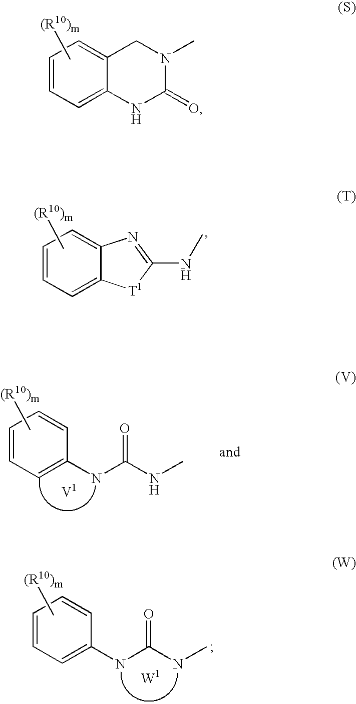
wherein T¹ is O, S, or NR⁵, wherein R⁵ is selected from hydrogen, C₁₋₈ alkyl, substituted C₁₋₈ alkyl, C₃₋₇ cycloalkyl, and heterocyclyl; and V¹ and W¹ define an optionally substituted five-to-six membered heterocyclic ring; provided that when Uc is T and T¹ is S, then at least one of R³ and R⁴ is not hydrogen, and further provided that when both X and Y are N, Uc is not T;
R⁹ is attached to any available carbon atom of the piperidinyl or piperazinyl ring and is selected from the group consisting of hydroxy, lower alkoxy, oxo (═O), halogen, cyano, halo C₁₋₄ alkyl, halo C₁₋₄ alkoxy, and C₁₋₄ lower alkyl optionally substituted with one to two R¹⁵;
R^{9a} and R^{9b} are selected from the group consisting of hydrogen and C₁₋₈ alkyl optionally substituted with one to two R¹⁵;
R¹⁰ is attached to any available carbon atom of the benzo or phenyl ring and at each occurrence is independently selected from the group consisting of C₁₋₈ alkyl, substituted C₁₋₈ alkyl, hydroxy, C₁₋₈ alkoxy, halogen, cyano, C₁₋₈ haloalkoxy, amino, alkylamino, heterocyclyl, heteroaryl, C₃₋₇ cycloalkyl, or phenyl said heterocyclyl, heteroaryl, C₃₋₇ cycloalkyl and phenyl bring optionally substituted by one to three substituents independently selected from R¹⁶;
R¹⁵ at each occurrence is independently selected from the group consisting of hydroxy, C₁₋₄ alkoxy, halo, cyano, trifluoromethyl, trifluoromethoxy, amino, and alkylamino;
R¹⁶ at each occurrence is independently selected from the group consisting of C₁₋₄ alkyl, hydroxy, C₁₋₄ alkoxy, halo, cyano, trifluoromethyl, trifluoromethoxy, amino, and alkylamino;
m is 0, 1, 2, 3, or 4;
n is 0 or 1;
p is 0, 1, 2, 3 or 4; and,
pharmaceutically-acceptable salts thereof.

2. A compound according to claim 1 wherein U^{c} is T, and R⁴ is methyl, ethyl, 1-methylethyl, isopropyl, 1-hydroxyethyl or 2-hydroxyethyl.

3. A compound according to claim 1 wherein:
Ar is pyrimidinyl or optionally-substituted phenyl;
Q is CH₂;
R² is hydrogen;
R³ and R⁴ are, independently selected from the group consisting of hydrogen, alkyl, hydroxyalkyl, and alkoxyalkyl;
R⁹ is selected from the group consisting of methyl, ethyl, hydroxy, methoxy, oxo (═O), halo, and cyano;
R^{9a} and R^{9b} are independently selected from the group consisting of hydrogen, methyl and ethyl;
n is 1; and
p is 0 or 1.

4. A compound according to claim 1 wherein X is N and Y is CR^{9b}.

5. A compound according to claim 1 wherein X and Y are both N.

6. A compound according to claim 1 wherein X is N⁺R^{9a} Z⁻, and Y is CR^{9b}.

7. A compound according to claim 1 wherein
U_{c} is IIIa. 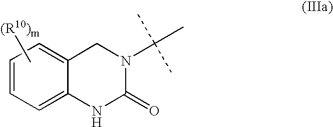
R¹⁰ is selected from C₁₋₄ alkyl, halogen, cyano, and C₁₋₄ alkoxy; and
m is 0, 1, or 2.

8. A compound according to claim 1 wherein:
U_{c} optionally substituted 2-aminobenzoxazole (IIIb); 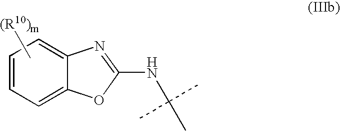
R¹⁰ is selected from lower alkyl, halogen, cyano, and lower alkoxy; and
m is 0, 1, or 2.

9. A compound according to claim 8wherein:
Ar is phenyl or pyrimidinyl, either optionally substituted with one, two, or three groups selected from the group consisting of halo, alkyl, heteroalkyl, alkoxy, nitro, trifluoromethyl, alkylsulfonyl, and optionally-substituted phenyl;
Q is CH₂;
R² and R³ are hydrogen;
R⁴ is methyl, ethyl, 1-methylethyl, isopropyl, 1-hydroxyethyl or 2-hydroxyethyl; and
R⁹ is selected from the group consisting of C₁₋₄alkyl, oxo (═O), halogen, and hydroxy.

10. A compound according to claim 1 wherein:
U_{c} is IIIc; 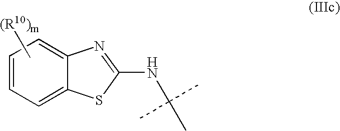
R¹⁰ is selected from the group consisting of C₁₋₄ alkyl, halogen, cyano, and C₁₋₄ alkoxy; and,
m is 0, 1, or 2.

11. A compound according to claim 10 wherein:
R² and R³ are hydrogen; and
R⁴ is methyl, ethyl, 1-methylethyl, isopropyl, 1-hydroxyethyl or 2-hydroxyethyl.

12. A compound according to claim 1 wherein:
U^{c} is IIId; 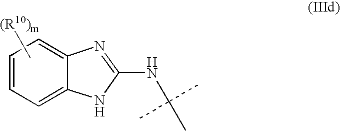
R¹⁰ is selected from the group consisting of C₁₋₄ alkyl, halogen, cyano, and C₁₋₄ alkoxy; and,
m is 0, 1, or 2.

13. A compound according to claim 1 wherein:
U_{c} is IIIe; 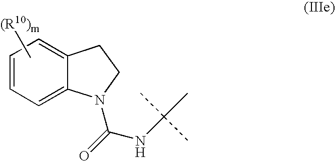
R₁₀ is selected from the group consisting of C₁₋₄ alkyl, halogen, cyano, and C₁₋₄ alkoxy; and,
m is 0, 1, or 2.

14. A compound according to claim 1 wherein:
U_{c} is IIIf; 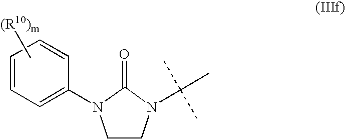
R¹⁰ is selected from the group consisting of C₁₋₄ alkyl, halogen, cyano, and C₁₋₄ alkoxy; and,
m is 0, 1, or 2.

15. A compound according to claim 1 having the Formula Ia: 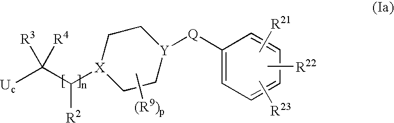
wherein,
X is N or N⁺R^{9a} Z⁻; Y is CR^{9a} or N;
Z is a pharmaceutically acceptable anion;
R² and R³ are hydrogen;
R^{9a} is lower alkyl and optionally present;
R²¹, R²², and R²³ are attached to any available carbon atom of the phenyl ring and are independently selected from the group consisting of hydrogen, lower alkyl, lower alkoxy, halogen, cyano, trifluoromethyl, trifluoromethoxy, C₁₋₄alkylsulfonyl, amino, and alkylamino; and
n is 1.

16. A compound according to claim 15 wherein Q is CH₂.

17. A compound according to claim 15 wherein:
R²¹, R²², and R²³, and the phenyl ring to which they are attached, form 4-chlorophenyl or 3,4-dichlorophenyl;
R⁴ is methyl, ethyl, 1-methylethyl, isopropyl, 1-hydroxyethyl or 2-hydroxyethyl; and
p is 0 or 1.

18. A compound according to claim 16 in which U^{c} is selected from one of, 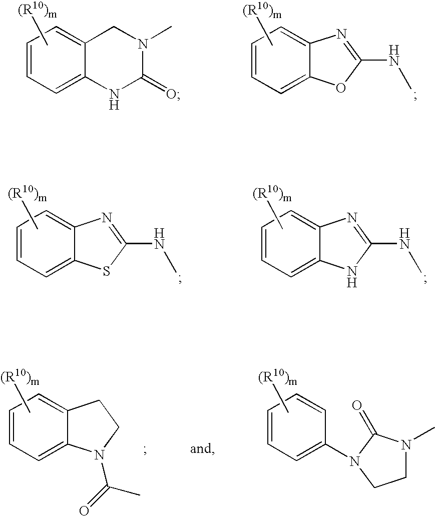
wherein:
R¹⁰ is selected from the group consisting of C₁₋₄ alkyl, halogen, cyano, and C₁₋₄ alkoxy; and
m is 0, 1, or 2.

19. A pharmaceutical composition comprising a therapeutically effective amount of a compound according to claim 1 in admixture with at least one pharmaceutically-acceptable diluent, excipient or carrier.

20. A method of treatment of a disease selected from the group consisting of Crohn's disease, ulcerative colitis, asthma, hypersensitivity pneumonitis, eosinophilic pneumonias, rhinitis, psoriasis, dermatitis and eczema in a mammal comprising administering a therapeutically effective amount of a CCR-3 antagonist according to claim 1.

21. The method of claim 20, wherein the disease is asthma.
